(19) United States
(12) Patent Application Publication
(10) Pub. No.: US 2010/0130512 A1
(43) Pub. Date: May 27, 2010

(54) FUSED-RING HETEROCYCLE OPIOIDS

(75) Inventor: Mark P. Wentland, Menands, NY (US)

Correspondence Address: HESLIN ROTHENBERG FARLEY & MESITI PC, 5 COLUMBIA CIRCLE, ALBANY, NY, 12203, US

(73) Assignee: RENSSELAER POLYTECHNIC INSTITUTE, Troy, NY (US)

(21) Appl. No.: 12/599,174

(22) PCT Filed: May 15, 2008
(86) PCT No.: PCT/US08/63713

§ 371 Date: Dec. 7, 2009

Related U.S. Application Data
(60) Provisional application No. 60/938,242, filed on May 16, 2007.

(51) Int. Cl. A61K 31/529; C07D 471/08; C07D 471/00; C07D 489/12; C07D 489/00; A61K 31/439; A61K 31/4748; A61K 31/485; A61P 17/04; A61P 1/12; A61P 1/00; A61P 3/04; A61P 11/00; A61P 25/30; A61P 25/04

(52) U.S. Cl. 514/257; 544/247; 546/63; 546/26; 514/286; 514/279

(57) ABSTRACT

Compounds of formula:

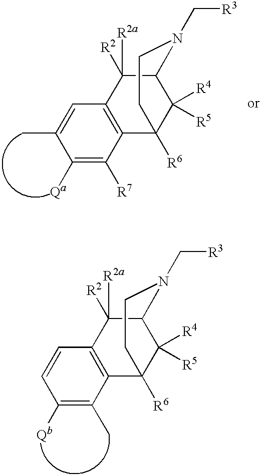

are disclosed. In these compounds

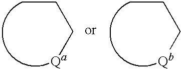

is a heterocyclic ring. The compounds are useful as analgesics, anti-pruritics, anti-diarrheal agents, anticonvulsants, antitussives, anorexics/antiobesity agents and as treatments for hyperalgesia, drug addiction, respiratory depression, dyskinesia, pain (including neuropathic pain), irritable bowel syndrome and gastrointestinal motility disorders.

### FEDERALLY SPONSORED RESEARCH

The following invention was made with Government support under contract number R01 DA12180 awarded by U.S. Dept of Health and Human Services. The Government has certain rights in this invention.

### FIELD OF THE INVENTION

Invention relates to opioid receptor binding compounds containing a heterocyclic moiety. The compounds are useful as analgesics, anti-diarrheal agents, anticonvulsants, anti-obesity agents, antitussives, anti-cocaine, and anti-addiction medications.

### BACKGROUND OF THE INVENTION

Opiates have been the subject of intense research since the isolation of morphine in 1805, and thousands of compounds having opiate or opiate-like activity have been identified. Many opioid receptor-interactive compounds including those used for producing analgesia (e.g., morphine) and those used for treating drug addiction (e.g., naltrexone and cyclazocine) in humans have limited utility due to poor oral bioavailability and a very rapid clearance rate from the body. This has been shown in many instances to be due to the presence of the 8-hydroxyl group (OH) of 2,6-methano-3-benzazocines, also known as benzomorphans [(e.g., cyclazocine and EKC (ethylketocyclazocine)] and the corresponding 3-OH group in morphinanes (e.g., morphine).

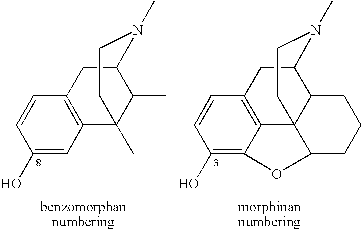

The high polarity of these hydroxyl groups retards oral absorption of the parent molecules. Furthermore, the 8-(or 3-)OH group is prone to sulfonation and glucuronidation (Phase II metabolism), both of which facilitate rapid excretion of the active compounds, leading to disadvantageously short half-lives for the active compounds. Until the publications of Wentland in 2001, the uniform experience in the art of the past seventy years had been that removal or replacement of the 8-(or 3-) OH group had led to pharmacologically inactive compounds.

U.S. Pat. No. 6,784,187 (to Wentland) disclosed that the phenolic OH of opioids could be replaced by CONH₂. In the cyclazocine series of opioids, it was shown that 8-carboxamidocyclazocine (8-CAC) had high affinity for ∥ and κ opioid receptors. In studies in vivo, 8-CAC showed high antinociception activity and a much longer duration of action than cyclazocine (15 h vs. 2 h) when both were dosed at 1 mg/kg ip in mice. Preliminary structure-activity relationship studies for 8-CAC revealed that mono-substitution of the carboxamide nitrogen with methyl or phenyl reduced binding affinity for guinea pig μ receptors 75- and 2313-fold, respectively whereas dimethylation of the carboxamide group reduced binding affinity 9375-fold. The finding that substitution of the carboxamide nitrogen had such a detrimental effect with these groups suggested that the NH₂ of the amide was critical to opioid binding.

### SUMMARY OF THE INVENTION

We have now found that the 8-position can be cyclized back into the aromatic ring at either 7 or 9 to provide compounds that exhibit excellent opioid binding and, presumably, good metabolic stability. The compounds of the invention are therefore useful as analgesics, anti-pruritics, anti-diarrheal agents, anticonvulsants, antitussives, anorexics and as treatments for hyperalgesia, drug addiction, respiratory depression, dyskinesia, pain (including neuropathic pain), irritable bowel syndrome and gastrointestinal motility disorders. Drug addiction, as used herein, includes alcohol and nicotine addiction. There is evidence in the literature that the compounds may also be useful as immunosuppressants and antiinflammatories and for reducing ischemic damage (and cardioprotection), for improving learning and memory, and for treating urinary incontinence. Those species that do not cross the blood-brain barrier are also useful for treating opioid-induced constipation and urinary retention.

In one aspect, the invention relates to compounds of formula:

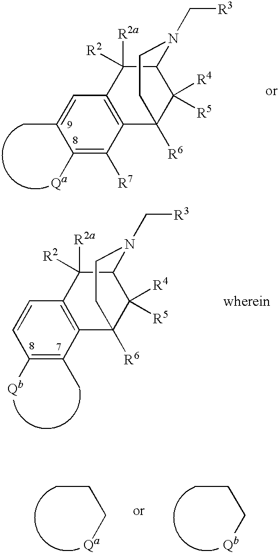

is a heterocyclic ring, which may be substituted or further fused to form a residue of one to three rings;

Q^{a} is chosen from

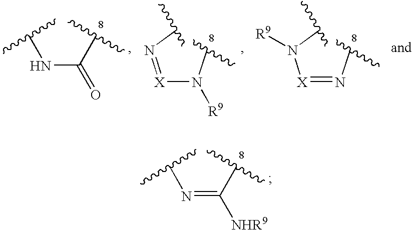

Q^{b} is chosen from

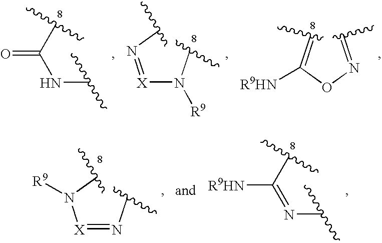

X is N or CR⁹;

R² and R^{2a} are both hydrogen or taken together R² and R^{2a} are ═O;

R³ is chosen from hydrogen, (C₁-C₈)hydrocarbon, heterocyclyl, heterocyclylalkyl and hydroxyalkyl;

R⁴ is chosen from hydrogen, hydroxy, amino, (C₁-C₆)alkoxy, (C₁-C₂₀)alkyl and (C₁-C₂₀)alkyl substituted with hydroxy or carbonyl;

R⁵ is (C₁-C₆)alkyl;

R⁶ is (C₁-C₆)alkyl;

R⁷ is chosen from hydrogen, NHR⁹ and hydroxy; or together R⁴, R⁵, R⁶ and R⁷ may form from one to three rings, said rings having optional additional substitution;

R⁹ is independently in each of its occurrences H, alkyl or

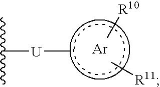

U is (CH₂)ₙ, wherein one or more CH₂ may be replaced by —O—, cycloalkyl or —CR^{1a}R^{1b};

R^{1a} and R^{1b} are chosen independently from hydrogen, halogen, (C₁-C₆)alkyl, (C₁-C₆)alkoxy and (C₁-C₆)alkylthio;

Ar is an aryl or heteroaryl residue of one to three rings;

R¹⁰ is one or two residues chosen independently from hydrogen, hydroxyl, halogen, (C₁-C₆)alkyl, (C₁-C₆)alkoxy, halo(C₁-C₆)alkyl and halo(C₁-C₆)alkoxy and (C₁-C₆)alkylthio;

R¹¹ is H or

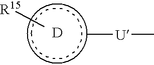

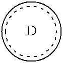

is an aryl or heteroaryl residue of one to three rings;

U′ is (CH₂)ₘ, wherein one or more CH₂ may be replaced by —O—, cycloalkyl, —CR^{1a}R^{1b}, —C(═O)— or —NH—;

R¹⁵ is one or two residues chosen independently from hydrogen, hydroxyl, halogen, (C₁-C₆)alkyl, (C₁-C₆)alkoxy, halo(C₁-C₆)alkyl and halo(C₁-C₆)alkoxy and (C₁-C₆)alkylthio;

m is zero or an integer from 1 to 6; and

n is an integer from 1 to 6.

Subclasses of the foregoing structure include:

A) 2,6-methano-3-benzazocines of the structure shown above, in which R⁴, R⁵, R⁶ and R⁷ do not form additional rings:

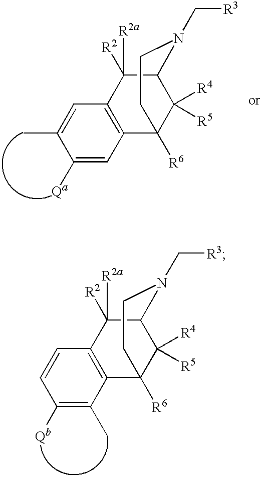

B) morphinans in which R⁵ and R⁶ form one ring:

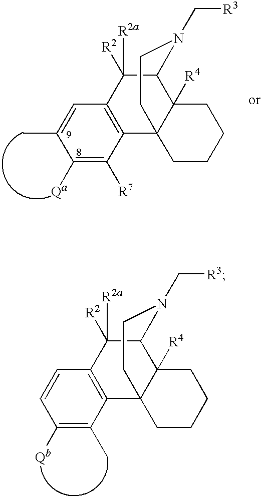

C) morphinans in which R⁵, R⁶ and R⁷ form two rings:

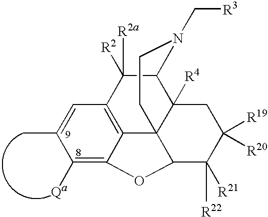

in which

R⁴ is hydrogen, hydroxy, amino or (C₁-C₆)alkoxy;

R¹⁹ is hydrogen or (C₁-C₆)alkyl;

R²⁰ is chosen from hydrogen, (C₁-C₆)alkyl and hydroxy((C₁-C₆)alkyl); or together, R¹⁹ and R²⁰ form a spiro-fused carbocycle of 5 to 10 carbons;

R²¹ is hydrogen;

R²² is chosen from hydroxy, (C₁-C₆)alkoxy and —NR¹³R¹⁴; or together, R²¹ and R²² form a carbonyl or a vinyl substituent;

D) morphinans in which R⁵, R⁶ and R⁷ form two rings and the nitrogen is quaternized:

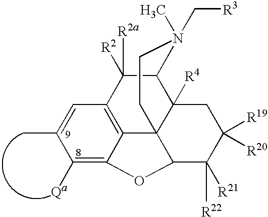

in which

R⁴ is hydrogen, hydroxy, amino or (C₁-C₆)alkoxy;

R¹⁹ is hydrogen or (C₁-C₆)alkyl;

R²⁰ is chosen from hydrogen, (C₁-C₆)alkyl and hydroxy((C₁-C₆)alkyl); or together, R¹⁹ and R²⁰ form a spiro-fused carbocycle of 5 to 10 carbons;

R²¹ is hydrogen;

R²² is chosen from hydroxy, (C₁-C₆)alkoxy and —NR¹³R¹⁴; or together, R²¹ and R²² form a carbonyl or a vinyl substituent; and E- is a pharmaceutically acceptable anion; and

E) morphinans wherein R⁴ and R¹¹ form an additional sixth ring, which may be saturated or unsaturated:

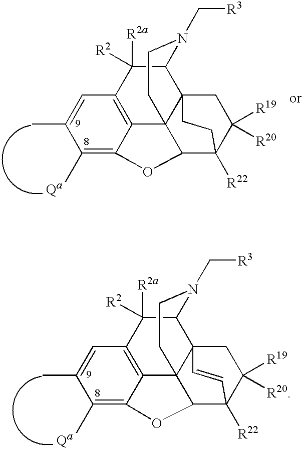

In another aspect, the invention relates to a compound of formula

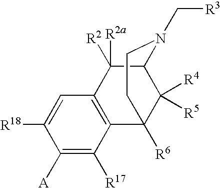

wherein

A is chosen from —C(═O)NR⁹R¹² and —C(═S)NR⁹R¹²;

R² and R^{2a} are both hydrogen or taken together R² and R^{2a} are ═O;

R³ is chosen from hydrogen, (C₁-C₈)hydrocarbon, heterocyclyl, heterocyclylalkyl and hydroxyalkyl;

R⁴ is chosen from hydrogen, hydroxy, amino, (C₁-C₆)alkoxy, (C₁-C₂₀)alkyl and (C₁-C₂₀)alkyl substituted with hydroxy or carbonyl;

R⁵ is (C₁-C₆)alkyl;

R⁶ is (C₁-C₆)alkyl;

or together R⁴, R⁵ and R⁶ may form from one to three rings, said rings having optional additional substitution;

R⁹ in each of its occurrences is independently chosen from H, alkyl and

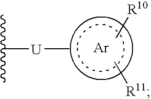

U is (CH₂)ₙ, wherein one or more CH₂ may be replaced by —O—, cycloalkyl or —CR^{1a}R^{1b};

R^{1a} and R^{1b} are chosen independently from hydrogen, halogen, (C₁-C₆)alkyl, (C₁-C₆)alkoxy and (C₁-C₆)alkylthio;

Ar is an aryl or heteroaryl residue of one to three rings;

R¹⁰ is one or two residues chosen independently from hydrogen, hydroxyl, halogen, (C₁-C₆)alkyl, (C₁-C₆)alkoxy, halo(C₁-C₆)alkyl and halo(C₁-C₆)alkoxy and (C₁-C₆)alkylthio;

R¹¹ is H or

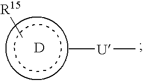

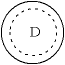

is an aryl or heteroaryl residue of one to three rings;

U′ is (CH₂)ₘ, wherein one or more CH₂ may be replaced by —O—, cycloalkyl, —CR^{1a}R^{1b}, —C(═O)— or —NH—;

R¹² is chosen from hydrogen and (C₁-C₆)alkyl;

R¹⁵ is one or two residues chosen independently from hydrogen, hydroxyl, halogen, (C₁-C₆)alkyl, (C₁-C₆)alkoxy, halo(C₁-C₆)alkyl and halo(C₁-C₆)alkoxy and (C₁-C₆)alkylthio;

one of R¹⁷ or R¹⁸ is NHR⁹ and the other is hydrogen;

m is zero or an integer from 1 to 6; and

n is an integer from 1 to 6.

In another aspect the invention relates to a pharmaceutical formulation comprising a pharmaceutically acceptable carrier and a compound as described above.

In another aspect the invention relates to a method for treating a disease or condition by altering a response mediated by an opioid receptor comprising bringing into contact with said opioid receptor a compound as described above.

### DETAILED DESCRIPTION OF THE INVENTION

From many years of SAR studies, it is known that the hydroxyl of morphinans and benzomorphans interacts with a specific site in the opiate receptor. We have now surprisingly found that the hydroxyl can be replaced with a heterocycle fused to the aromatic ring through a carbon that occupies the position formerly occupied by the hydroxyl. A fairly wide range of fused heterocycles exhibit binding to at least one of the opioid binding sites (μ, δ or κ) in the desired range below 250 nanomolar.

In one aspect the invention relates to compounds of formula

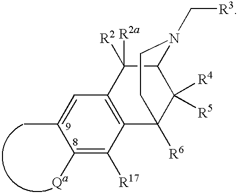

This subgenus comprises compounds in which the heterocyclic ring is fused at the 8,9-positions of the benzazocine. In certain embodiments

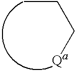

is chosen from

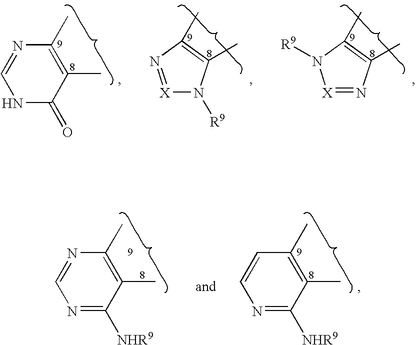

and the compounds are of the formulae:

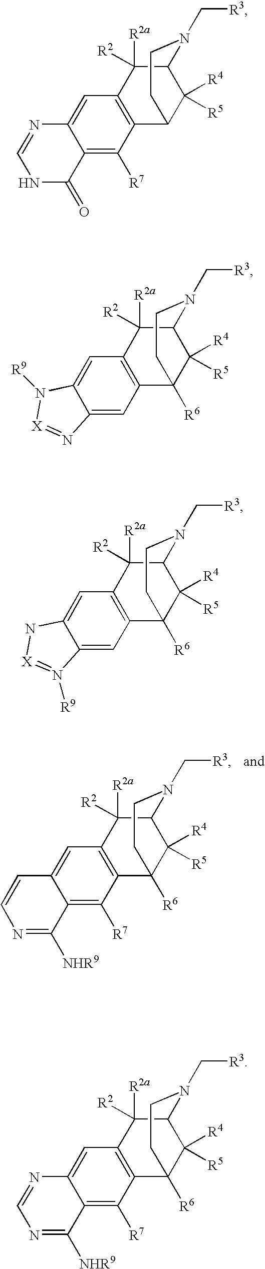

In another aspect the invention relates to compounds of formula

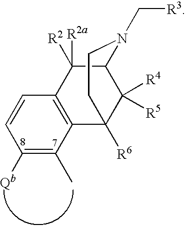

This subgenus comprises compounds in which the heterocyclic ring is fused at the 7,8-positions of the benzazocine. In certain embodiments

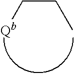

is chosen from

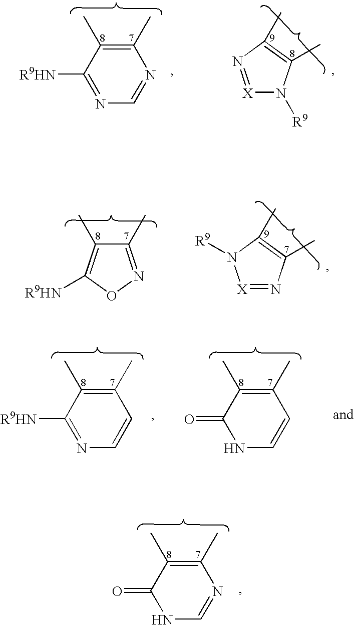

and the compounds are of the formulae:

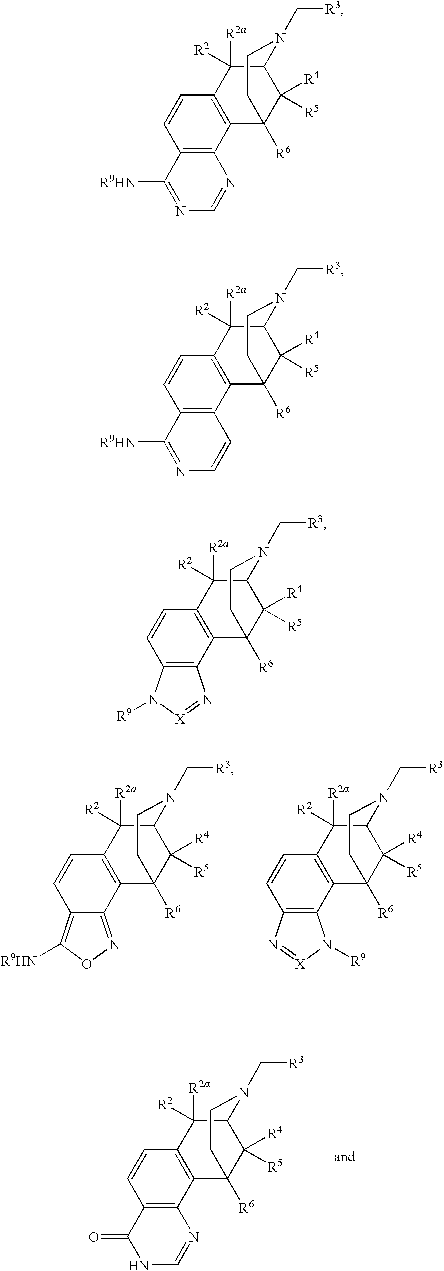

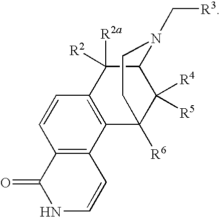

In another aspect, the invention relates to compounds of formula

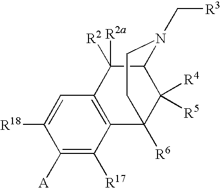

in which A is —C(═O)NR⁹R¹² or —C(═S)NR⁹R¹². These compounds are useful both as intermediates in the synthesis of 7,8-fused pyrimidines and in their own right as opioid receptor binding compounds (see example 6 in Table 1 below). Commonly, R¹² will be hydrogen.

In one major subclass, the groups R⁹ are biphenyls, diaryl ethers and the like. Illustrative formulae are:

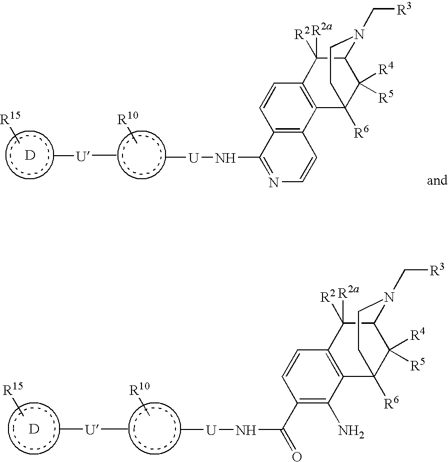

Preferred values of R⁹are hydrogen and those in which
(a)

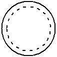

is phenyl, R¹⁰ is hydrogen and R¹¹ is

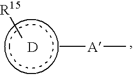

so that R¹¹ represents pyridinyl, phenyl, halophenyl, methylphenyl, methoxyphenyl (in all of which A′ is a direct bond) and phenoxy (in which A′ is —O—).
(b)

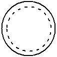

is chosen from phenyl, naphthyl, fluorenyl, carbazole, dibenzofuran and dibenzothiophene, R¹⁰ is hydrogen, methoxy, halogen or methyl; and R¹¹ is hydrogen;
(c)

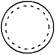

is pyridinyl, R¹⁰ is hydrogen and R¹¹ is chosen from phenyl, halophenyl, methylphenyl, methoxyphenyl and phenoxy.

It is known in the art that compounds that are μ, δ and κ agonists exhibit analgesic activity; compounds that are selective μ agonists exhibit anti-diarrheal activity and are useful in treating dyskinesia; μ antagonists and κ agonists are useful in treating heroin, cocaine, alcohol and nicotine addiction; κ agonists are also anti-pruritic agents and are useful in treating hyperalgesia. Recently it has been found [Peterson et al. Biochem. Pharmacol. 61, 1141-1151 (2001)] that κ agonists are also useful in treating retroviral infections. In general, the dextrorotatory isomers of morphinans of type III above are useful as antitussives and anticonvulsants.

Opioid receptor ligands having known high affinity are shown in the following charts. Attachment of a fused ring

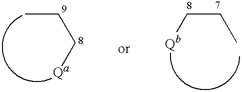

at the carbon carrying the phenolic OH (designated 8) and its adjacent carbon (designated 7 or 9) in these compounds produces compounds that exhibit opioid activity. As will be apparent to the artisan, the ring containing Qb will not be attached to morphinanes and similar compounds in Charts 2 and 3, which already possess substitution at C-7. Embodiments of the invention include each of the compounds set forth in the following charts in which the phenolic hydroxyl is replaced by a fused ring attached at the carbon to which the phenolic —OH is attached and the carbon adjacent thereto.

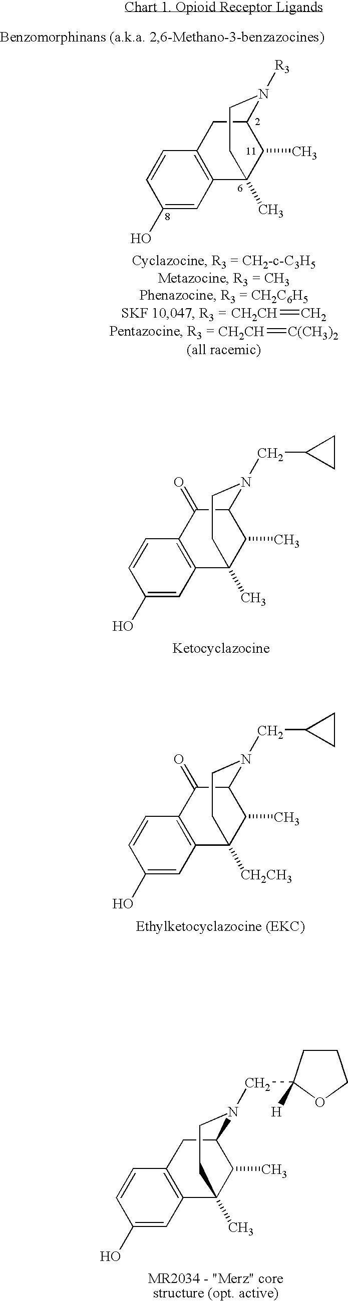

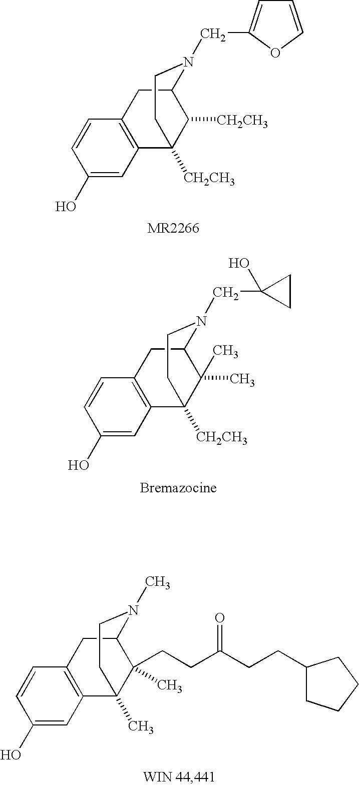

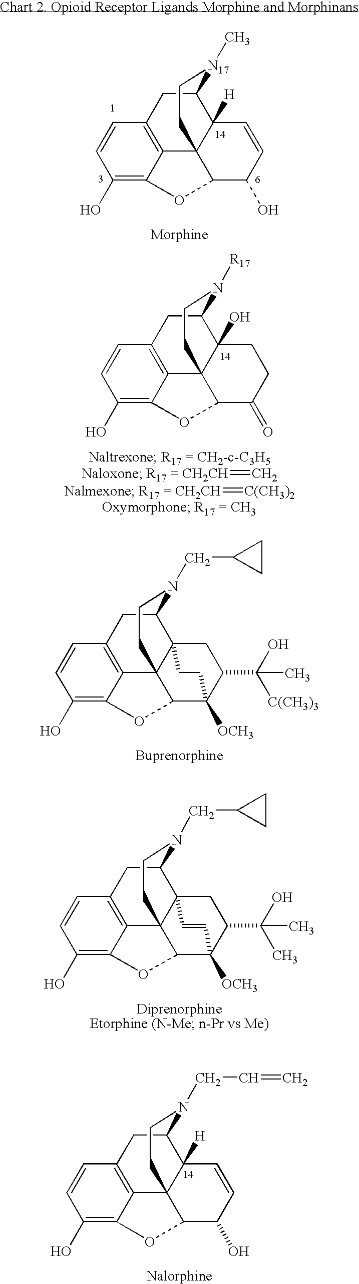

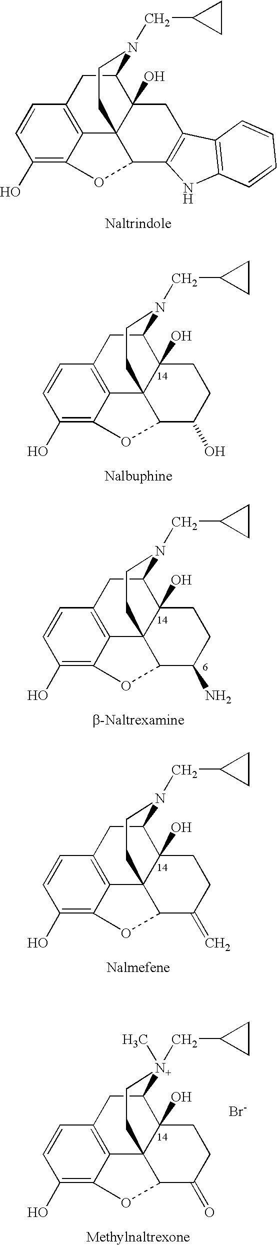

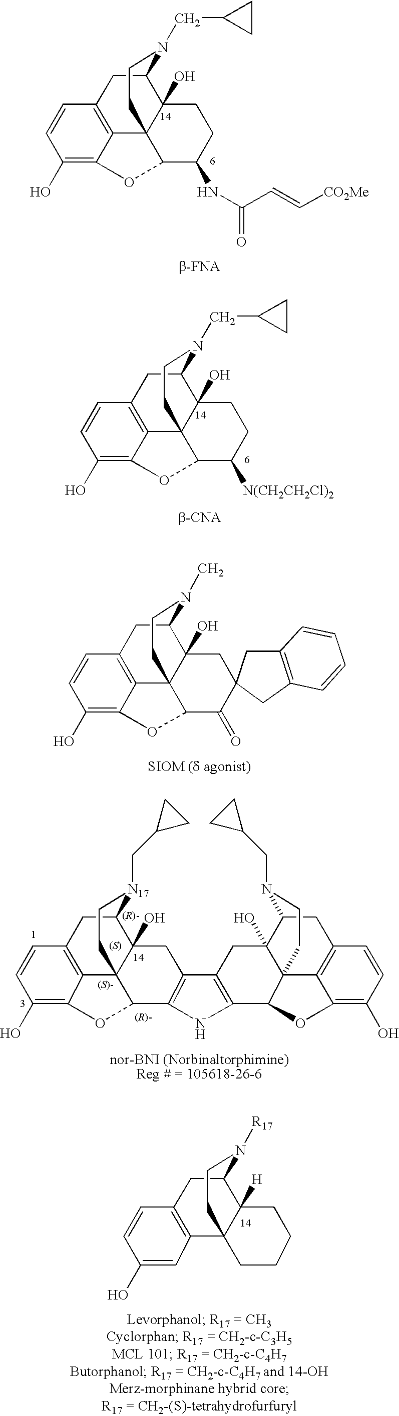

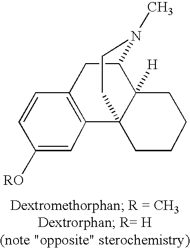

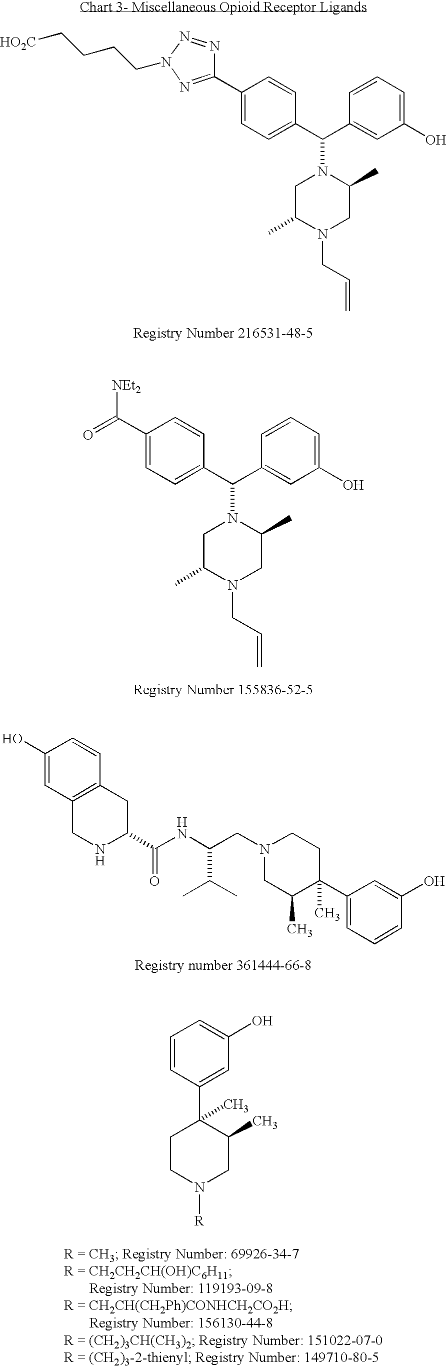

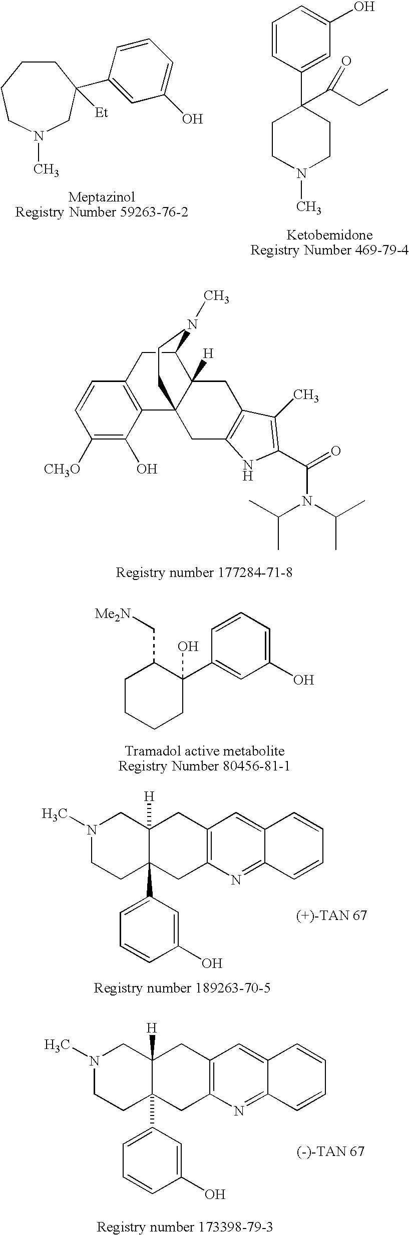

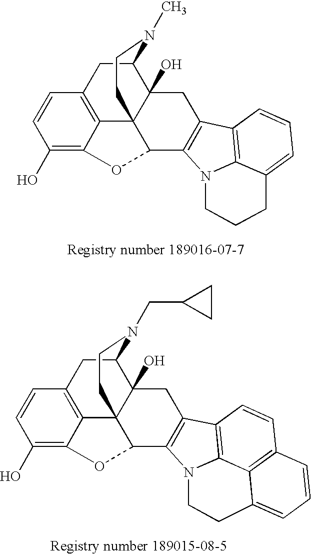

Other opioid receptor ligands are described in Aldrich, J. V. “Analgesics” in Burger's Medicinal Chemistry and Drug Discovery, M. E. Wolff ed., John Wiley & Sons 1996, pages 321-44, the disclosures of which are incorporated herein by reference.

We have examined the opioid receptor binding of a series of fused-ring analogs of known compounds that interact at opioid receptors. Binding assays used to screen compounds are similar to those previously reported by Neumeyer et al., Design and Synthesis of Novel Dimeric Morphinan Ligands for κ and μ Opioid Receptors. J. Med. Chem. 2003, 46, 5162. Membrane protein from CHO cells that stably expressed one type of the human opioid receptor were incubated with 12 different concentrations of the compound in the presence of either 1 nM [³H]U69,593¹⁰ (κ), 0.25 nM [³H]DAMGO¹¹ (μ) or 0.2 nM [³H]naltrindole¹² (δ) in a final volume of 1 mL of 50 mM Tris-HCl, pH 7.5 at 25° C. Incubation times of 60 min were used for [³H]U69,593 and [³H]DAMGO. Because of a slower association of [³H]naltrindole with the receptor, a 3 h incubation was used with this radioligand. Samples incubated with [³H]naltrindole also contained 10 mM MgCl₂ and 0.5 mM phenylmethylsulfonyl fluoride. Nonspecific binding was measured by inclusion of 10 μM naloxone. The binding was terminated by filtering the samples through Schleicher & Schuell No. 32 glass fiber filters using a Brandel 48-well cell harvester. The filters were subsequently washed three times with 3 mL of cold 50 mM Tris-HCl, pH 7.5, and were counted in 2 mL Ecoscint A scintillation fluid. For [³H]naltrindole and [³H]U69,593 binding, the filters were soaked in 0.1% polyethylenimine for at least 60 min before use. IC₅₀ values were-calculated by least squares fit to a logarithm-probit analysis. Kᵢ values of unlabeled compounds were calculated from the equation Kᵢ=(IC₅₀)/1+S where S=(concentration of radioligand)/(K_{d} of radioligand).¹³ Data are the mean±SEM from at least three experiments performed in triplicate.

[³⁵S]GTPγS Binding Assays. In a final volume of 0.5 mL, 12 different concentrations of each test compound were incubated with 15 μg (κ), 10 μg (δ) or 7.5 μg (μ) of CHO cell membranes that stably expressed either the human κ, δ or μ opioid receptor. The assay buffer consisted of 50 mM Tris-HCl, pH 7.4, 3 mM MgCl₂, 0.2 mM EGTA, 3 μM GDP, and 100 mM NaCl. The final concentration of [³⁵S]GTPγS was 0.080 nM. Nonspecific binding was measured by inclusion of 10 μM GTPγS. Binding was initiated by the addition of the membranes. After an incubation of 60 min at 30° C., the samples were filtered through Schleicher & Schuell No. 32 glass fiber filters. The filters were washed three times with cold 50 mM Tris-HCl, pH 7.5, and were counted in 2 mL of Ecoscint scintillation fluid. Data are the mean Eₘₐₓ and EC₅₀ values±S.E.M. from at least three separate experiments, performed in triplicate. For calculation of the Eₘₐₓ values, the basal [³⁵S]GTPγS binding was set at 0%. To determine antagonist activity of a compound at the μ opioid receptors, CHO membranes expressing the μ opioid receptor, were incubated with 12 different concentrations of the compound in the presence of 200 nM of the μ agonist DAMGO. To determine antagonist activity of a compound at the κ opioid receptors, CHO membranes expressing the κ opioid receptor, were incubated with the compound in the presence of 100 nM of the κ agonist U50,488. To determine if a compound was an antagonist at δ receptors, CHO membranes expressing the δ receptor were incubated with 12 different concentrations of the test compound in the presence of 10 nM of the δ-selective agonist SNC 80.

### Examples

TABLE 1
Kᵢ(nM)

| Ex. # | [3H]DAMGO (μ) | [3H]Naltrindole (δ) | [3H]U69,593 (K) | GTP_{Y}S data |
|---|---|---|---|---|
| 6 | 0.55 ± 0.029 | 35 ± 0.036 | 0.70 ± 0.036 | MOR antagonist, KOR moderate |
| | | | | agonist |
| 7 | 88 ± 5.2 | 2000 ± 12 | 32 ± 1.7 | |
| 8 | 270 ± 33 | 2000 ± 49 | 160 ± 7.1 | |
| 9 | 170 ± 6.7 | 780 ± 32 | 12 ± 0.65 | |
| 10 | 0.55 ± 0.018 | 120 ± 8.5 | 1.0 ± 0.071 | MOR and KOR antagonist |
| 11 | 890 ± 39 | 47% inh. at 10 μM | 560 ± 23 | |
| 12 | 44 ± 0.76 | 1500 ± 68 | 240 ± 1.8 | |
| 13 | 88 ± 7.2 | 1000 ± 37 | 48 ± 2.3 | |
| 14 | 6.9 ± 0.33 | 52 ± 2.6 | 8.6 ± 1.5 | |
| 15 | 28 ± 1.9 | 410 ± 61 | 140 ± 4.4 | |
| 29 | 1.4 ± 0.043 | 46 ± 2.6 | 0.23 ± 0.009 | MOR antagonist; KOR agonist |
| 30 | 10 ± 1.0 | 51 ± 7.8 | 0.81 ± 0.19 | MOR antagonist; KOR agonist |
| 34 | 0.31 ± 0.050 | 5.1 ± 0.65 | 0.063 ± 0.0016 | MOR weak agonist/antagonist, KOR |
| | | | | and DOR agonist |
| 35 | 0.77 ± 0.051 | 18 ± 0.90 | 0.050 ± 0.002 | MOR agonist/weak antagonist; KOR |
| | | | | agonist |
| 36 | 60 ± 7.9 | 730 ± 8.3 | 12 ± 1.3 | |
| 43 | 0.75 ± 0.038 | 99 ± 10 | 0.65 ± 0.005 | |

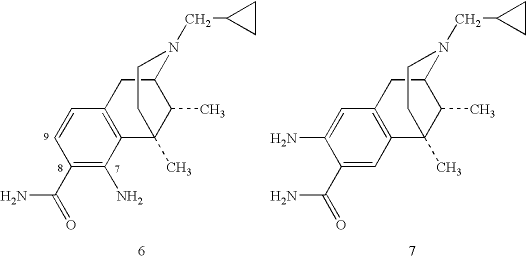

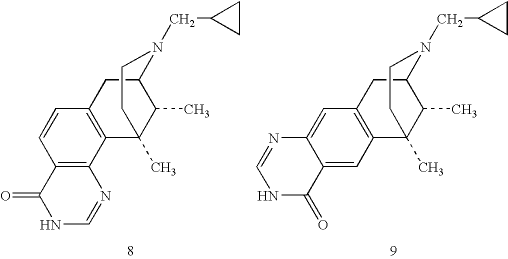

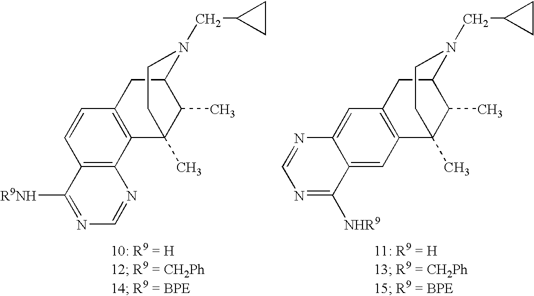

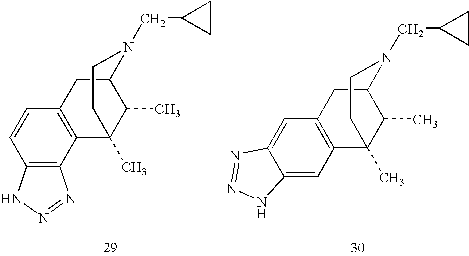

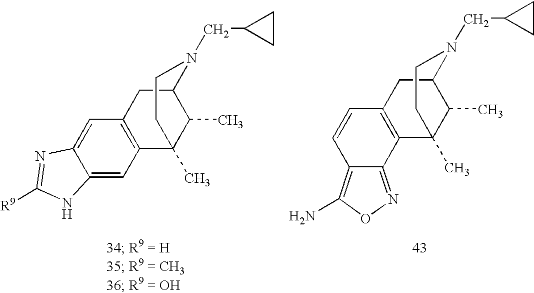

Antinociceptive activity is evaluated by the method described in Jiang et al. [J. Pharmacol. Exp. Ther. 264, 1021-1027 (1993), page 1022]. The ED_{50′}s of compounds of the invention are expected to be under 100 nmol in the mouse acetic acid writhing test when administered i.c.v., and an increase in the duration of action is expected for compounds of the invention compared to their “parents” when given by i.p. administration.

### DEFINITIONS

Throughout this specification the terms and substituents retain their definitions.

Alkyl is intended to include linear, branched, or cyclic hydrocarbon structures and combinations thereof. Lower alkyl refers to alkyl groups of from 1 to 6 carbon atoms. Examples of lower alkyl groups include methyl, ethyl, propyl, isopropyl, cyclopropyl, butyl, s-and t-butyl, cyclobutyl and the like. Preferred alkyl groups are those of C₂₀ or below. Cycloalkyl is a subset of alkyl and includes cyclic hydrocarbon groups of from 3 to 8 carbon atoms. Examples of cycloalkyl groups include c-propyl, c-butyl, c-pentyl, norbornyl and the like.

Alkoxy or alkoxyl refers to groups of from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons.

Aryl and heteroaryl mean a 5- or 6-membered aromatic or heteroaromatic ring containing 0-3 heteroatoms selected from O, N, or S; a bicyclic 9- or 10-membered aromatic or heteroaromatic ring system containing 0-3 heteroatoms selected from O, N, or S; or a tricyclic 13- or 14-membered aromatic or heteroaromatic ring system containing 0-3 heteroatoms selected from O, N, or S. The aromatic 6- to 14-membered carbocyclic rings include, e.g., benzene, naphthalene, indane, tetralin, and fluorene and the 5- to 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole.

Arylalkyl means an alkyl residue attached to an aryl ring. Examples are benzyl, phenethyl and the like. Heteroarylalkyl means an alkyl residue attached to a heteroaryl ring. Examples include, e.g., pyridinylmethyl, pyrimidinylethyl and the like.

Heterocycle means a cycloalkyl or aryl residue in which one to two of the carbons is replaced by a heteroatom such as oxygen, nitrogen or sulfur. Heteroaryls form a subset of heterocycles. Examples of heterocycles that fall within the scope of the invention include pyrrolidine, pyrazole, pyrrole, indole, quinoline, isoquinoline, 03tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like.

Substituted alkyl, aryl, cycloalkyl, or heterocyclyl refer to alkyl, aryl, cycloalkyl, or heterocyclyl wherein up to three H atoms in each residue are replaced with halogen, alkyl, aryl, cycloalkyl, heterocyclyl, hydroxy, lower-alkoxy, carboxy, carboalkoxy, carboxamido, cyano, carbonyl, —NO₂, —NR¹R²; alkylthio, sulfoxide, sulfone, acylamino, amidino, phenyl, benzyl, heteroaryl, phenoxy, benzyloxy, heteroaryloxy, or substituted phenyl, benzyl, heteroaryl, phenoxy, benzyloxy, or heteroaryloxy.

Virtually all of the compounds described herein contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, as well as their racemic and optically pure forms. In general it has been found that the levo isomer of morphinans and benzomorphans is the more potent antinociceptive agent, while the dextro isomer may be useful as an antitussive or antispasmodic agent. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included. For example, the structural representation

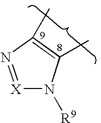

is intended to include both tautomers

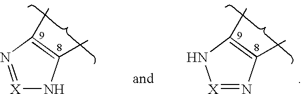

Some of the compounds of the invention are quaternary salts, i.e. cationic species. Therefore they will always be presented as salts, and the term “pharmaceutically acceptable salt” refers to salts whose counter ion (anion) derives from pharmaceutically acceptable non-toxic acids including inorganic acids, organic acids and water (which formally furnishes the hydroxide anion). Suitable pharmaceutically acceptable anions for the compounds of the present invention include hydroxide, acetate, benzenesulfonate (besylate), benzoate, bicarbonate, bisulfate, carbonate, camphorsulfonate, citrate, ethanesulfonate, fumarate, gluconate, glutamate, glycolate, bromide, chloride, isethionate, lactate, maleate, malate, mandelate, methanesulfonate, mucate, nitrate, pamoate, pantothenate, phosphate, succinate, sulfate, tartrate, trifluoroacetate, p-toluenesulfonate, acetamidobenzoate, adipate, alginate, aminosalicylate, anhydromethylenecitrate, ascorbate, aspartate, calcium edetate, camphorate, camsylate, caprate, caproate, caprylate, cinnamate, cyclamate, dichloroacetate, edetate (EDTA), edisylate, embonate, estolate, esylate, fluoride, formate, gentisate, gluceptate, glucuronate, glycerophosphate, glycolate, glycollylarsanilate, hexylresorcinate, hippurate, hydroxynaphthoate, iodide, lactobionate, malonate, mesylate, napadisylate, napsylate, nicotinate, oleate, orotate, oxalate, oxoglutarate, palmitate, pectinate, pectinate polymer, phenylethylbarbiturate, picrate, pidolate, propionate, rhodanide, salicylate, sebacate, stearate, tannate, theoclate, tosylate and the like. The desired salt may be obtained by ion exchange of whatever counter ion is obtained in the synthesis of the quat. These methods are well known to persons of skill. Although pharmaceutically acceptable counter ions will be preferred for preparing pharmaceutical formulations, other anions are quite acceptable as synthetic intermediates. Thus X may be pharmaceutically undesirable anions, such as iodide, oxalate, trifluoromethanesulfonate and the like, when such salts are chemical intermediates. When the compounds of the invention are bisquats, one may employ as counter ions either two monoanionic species (e.g. Cl₂) or a single dianionic species (e.g. fumarate). Similarly, one could employ oligoanionic species and make salts having appropriate ratios of quat to counterion, such as (quat)₃ citrates. These would be obvious equivalents.

### Abbreviations

The following abbreviations and terms have the indicated meanings throughout:
Ac=acetyl
BNB=4-bromomethyl-3-nitrobenzoic acid
Boc=t-butyloxy carbonyl
BPE=2(4-biphenylyl)ethyl=

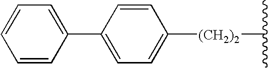

Bu=butyl
c-=cyclo
DAMGO=Tyr-ala-Gly-NMePhe-NHCH₂OH
DBU=diazabicyclo[5.4.0]undec-7-ene
DCM=dichloromethane=methylene chloride=CH₂Cl₂
DEAD=diethyl azodicarboxylate
DIC=diisopropylcarbodiimide
DIEA=N,N-diisopropylethyl amine
DMAP=4-N,N-dimethylaminopyridine
DMF=N,N-dimethylformamide
DMSO=dimethyl sulfoxide
DOR=delta opioid receptor
DPPF=1,1′-bis(diphenylphosphino)ferrocene
DVB=1,4-divinylbenzene
EEDQ=2-ethoxy-1-ethoxycarbonyl-1,2-dihydroquinoline
Fmoc=9-fluorenylmethoxycarbonyl
GC=gas chromatography
HATU=O-(7-Azabenzotriazol-1-yl)-1,1,3,3-tetramethyluronium hexafluorophosphate
HOAc=acetic acid
HOBt=hydroxybenzotriazole
KOR=kappa opioid receptor
Me=methyl
mesyl=methanesulfonyl
MOR=mu opioid receptor
MTBE=methyl t-butyl ether
NMO=N-methylmorpholine oxide
PEG=polyethylene glycol
Ph=phenyl
PhOH=phenol
PfP=pentafluorophenol
PPTS=pyridinium p-toluenesulfonate
PyBroP=bromo-tris-pyrrolidino-phosphonium hexafluorophosphate
rt=room temperature
sat'd=saturated
s-=secondary
t-=tertiary
TBDMS=t-butyldimethylsilyl
TFA=trifluoroacetic acid
THF=tetrahydrofuran
TMOF=trimethyl orthoformate
TMS=trimethylsilyl
tosyl=p-toluenesulfonyl
Trt=triphenylmethyl
U69,593=

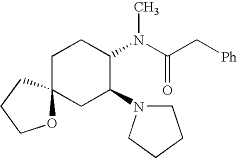

Terminology related to “protecting”, “deprotecting” and “protected” functionalities occurs throughout this application. Such terminology is well understood by persons of skill in the art and is used in the context of processes which involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is below, the person of ordinary skill can readily envision those groups that would be suitable as “protecting groups”. Suitable groups for that purpose are discussed in standard textbooks in the field of chemistry, such as Protective Groups in Organic Synthesis by T. W. Greene [John Wiley & Sons, New York, 1991], which is incorporated herein by reference.

The compounds of the invention are synthesized by one of the routes described below.

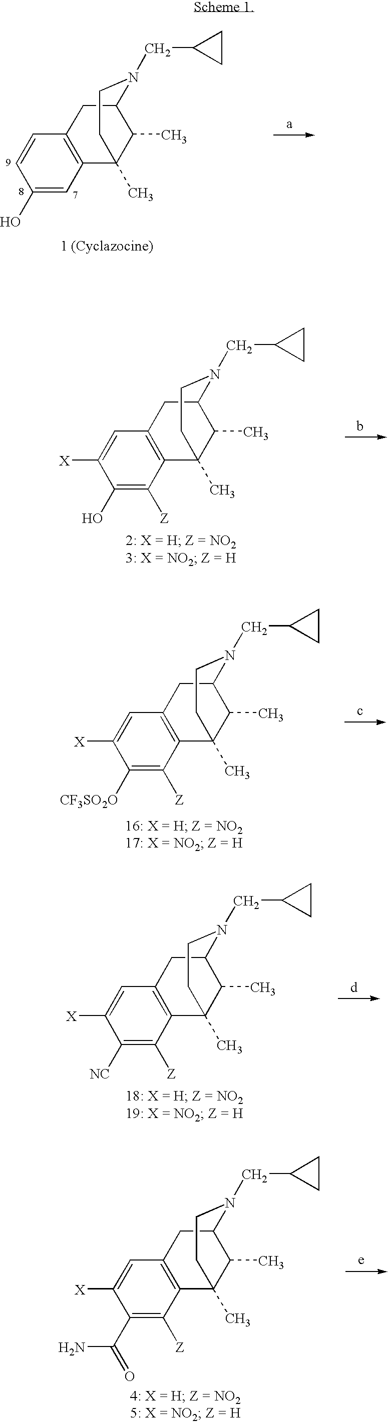

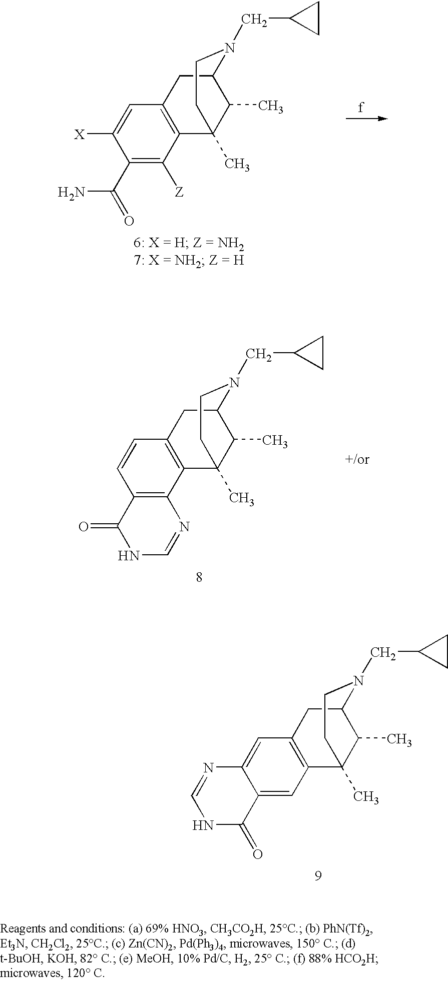

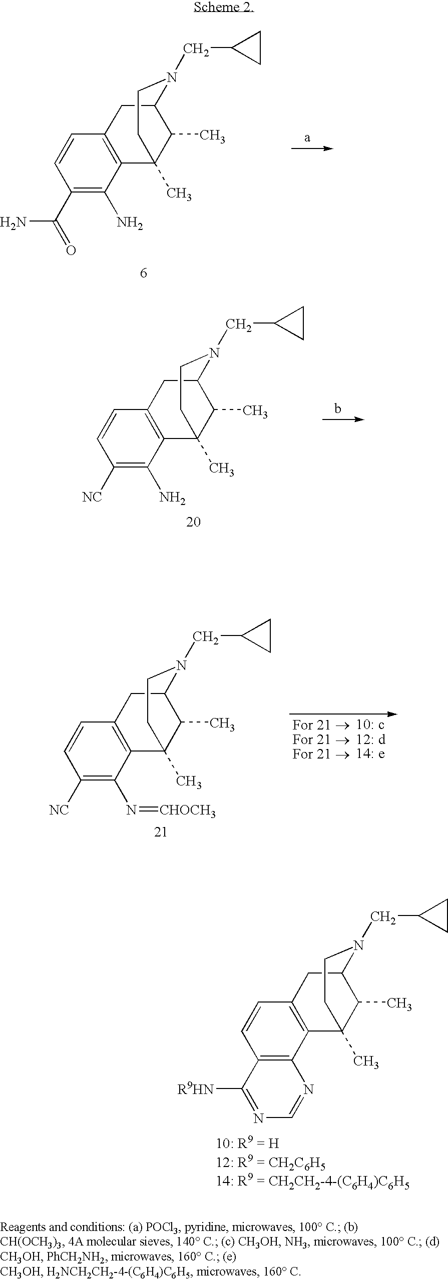

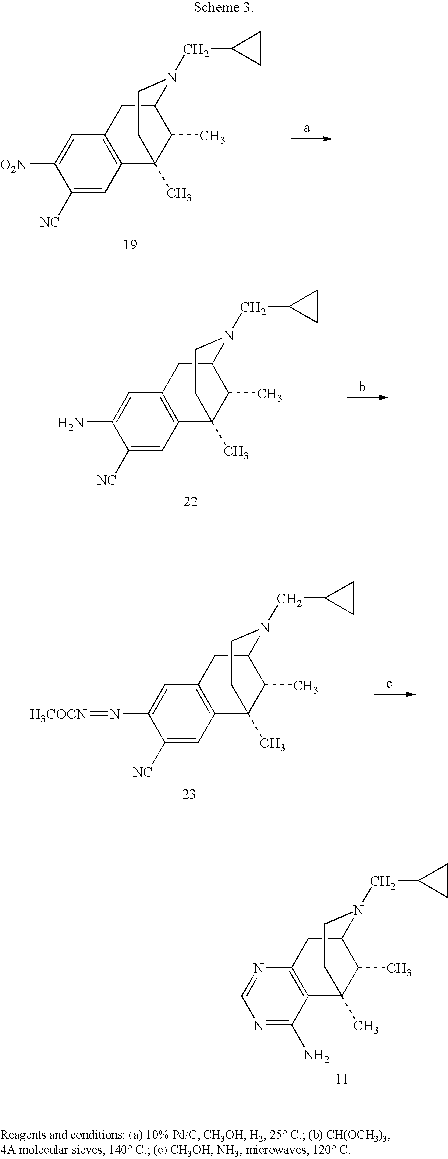

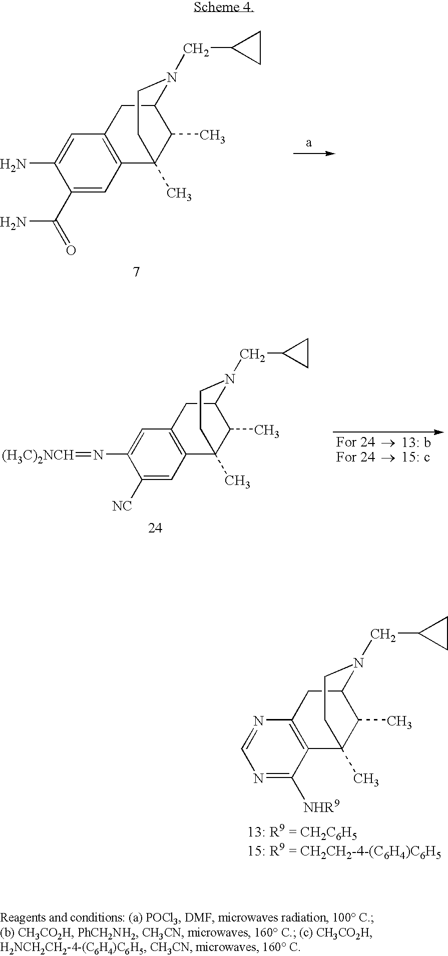

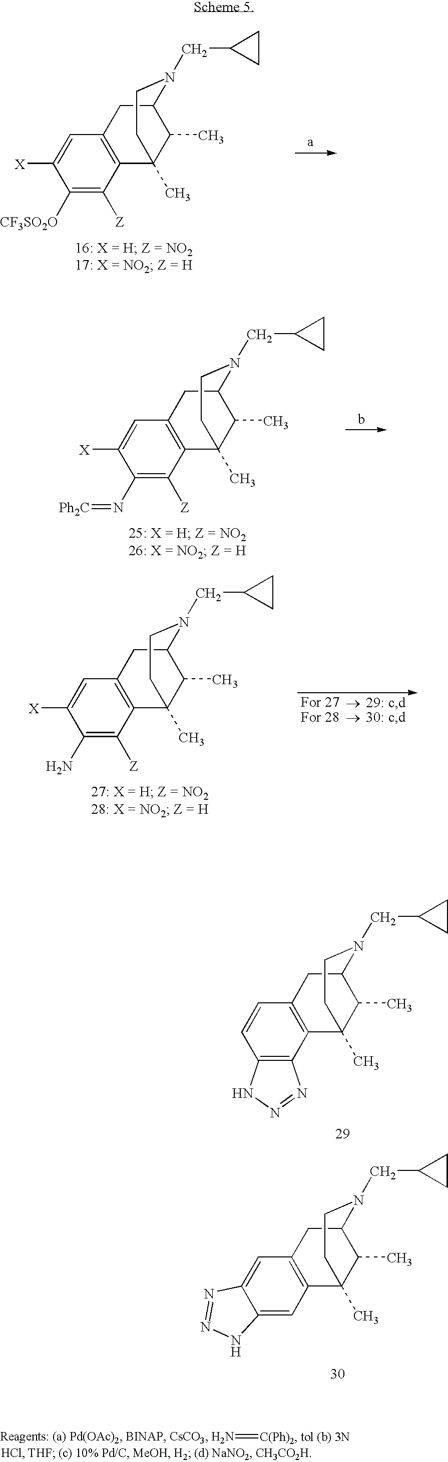

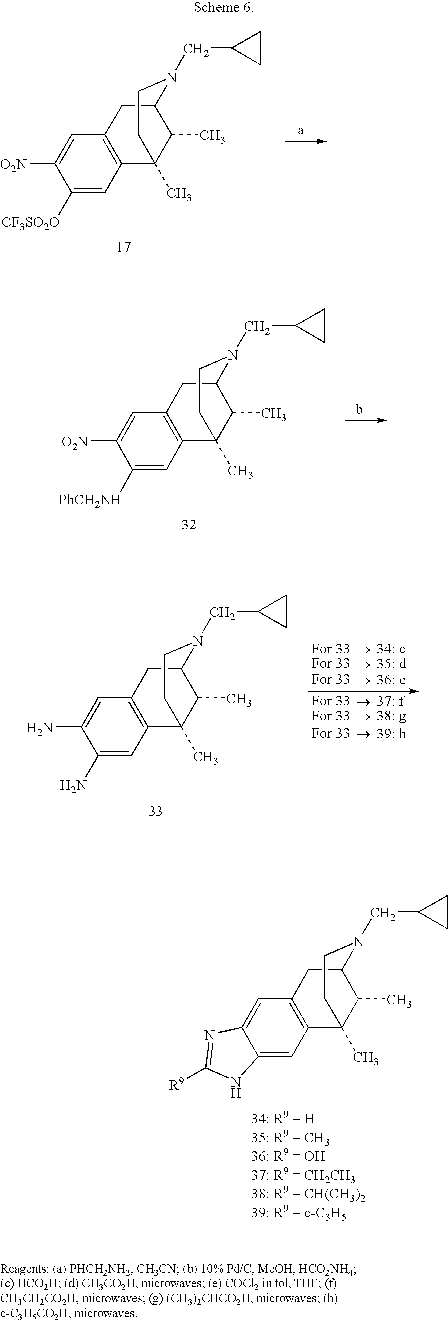

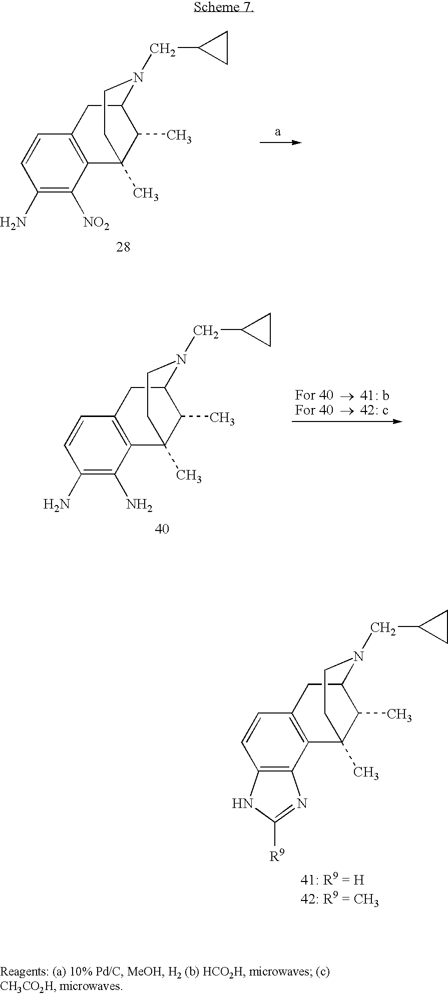

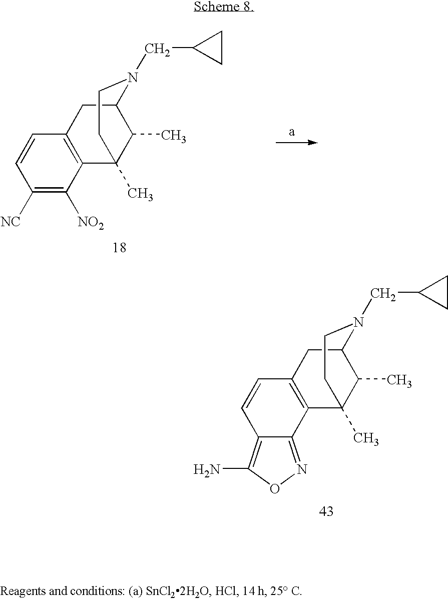

### Experimental Section

Proton NMR spectra and in certain cases ¹³C NMR were obtained on a Varian Unity-300 or 500 NMR spectrometer with tetramethylsilane as an internal reference for samples dissolved in CDCl₃. Samples dissolved in CD₃OD and DMSO-d₆ were referenced to the solvent. Proton NMR multiplicity data are denoted by s (singlet), d (doublet), t (triplet), q (quartet), m (multiplet), dd (doublet of doublets), and br (broad). Coupling constants are in hertz. Direct insertion probe chemical ionization mass spectral data were obtained on a Shimadzu GC-17A GC-MS mass spectrometer. Direct infusion electrospray ionization (in positively charged ion mode) mass spectral data were obtained on an Agilent 1100 series LC/MSD system (Germany). Melting points were determined on a Meltemp capillary melting point apparatus and were uncorrected. Infrared spectral data were obtained on a Perkin-Elmer Paragon 1000 FT-IR spectrophotometer. Optical rotation data was obtained from a Perkin-Elmer 241 polarimeter. The assigned structure of all test compounds and intermediates were consistent with the data. Carbon, hydrogen, and nitrogen elemental analyses for all novel targets were performed by Quantitative Technologies Inc., Whitehouse, N.J., and were within ±0.4% of theoretical values except as noted; the presence of water or other solvents was confirmed by proton NMR. Reactions were generally performed in an argon or nitrogen atmosphere. Commercially purchased chemicals were used without purification unless otherwise noted. The following reagents were purchased from Aldrich Chemical Company: N-hydroxysuccinimide, phenethylamine, 3-phenyl-1-propylamine, 4-aminobiphenyl, palladium acetate, 4-phenylbenzylamine and benzyl amine. The following reagent was purchased from Trans World Chemicals: 2-(4-biphenyl ethylamine). The following reagents were purchased from Strem Chemicals, Incorporated: 1,1′-bis(diphenyl-phosphino)ferrocene (dppf) and dichloro[1,1′-bis(diphenylphosphino)-ferrocene]palladium (II) dichloromethane adduct [PdCl₂(dppf)]. Pyridine was distilled from KOH. DMF and DMSO were distilled over CaH₂ under reduced pressure. Amines were purchased from Aldrich Chemical Company and used as received unless otherwise indicated. Toluene and Et₂O were distilled from sodium metal. THF was distilled from sodium/benzophenone ketyl. Pyridine was distilled from KOH. Methylene chloride was distilled from CaH₂. DMF and DMSO were distilled from CaH₂ under reduced pressure. Methanol was dried over 3± molecular sieves prior to use. Silica gel (Bodman Industries, ICN SiliTech 2-63 D 60A, 230-400 Mesh) was used for flash column chromatography.

Cis-(±)-3-(cyclopropylmethyl)-1,2,3,4,5,6-hexahydro-6,11-dimethyl-7-nitro-2,6-methano-3-benzazocine-8-ol (2) and cis-(±)-3-(cyclopropylmethyl)-1,2,3,4,5,6-hexahydro-6,11-dimethyl-9-nitro-2,6-methano-3-benzazocin-8-ol (3). A solution of 69% nitric acid (0.20 g) in 2.0 mL glacial acetic acid was added to a solution of cylazocine³ (1; 0.542 g, 2.0 mmol) in 3.0 mL glacial acetic acid at 25° C. After stirring at 25° C. for 3 h, tlc indicated the presence of starting material and an additional 0.10 gm of 69% nitric acid was added. After stirring 2 h at 25° C., tlc indicated all starting material was consumed and the reaction mixture was poured into a mixture of ice and excess concentrated ammonium hydroxide. The mixture was treated with ethyl acetate and the organic phase was washed with brine, dried over Na₂SO₄, filtered, and concentrated to give a crude solid produce which was purified by gradient silica gel flash chromatography (CH₂Cl₂:CH₃OH; 20:1→10:1) to give 2 (0.26 g, 40%) as an off-white solid and 3 (0.35 g, 54%) as an off-white foam: Recrystallization from MeOH/CH₂Cl₂ gave off-white crystals having mp 145° C. and mp 175° C., respectively.

For 2: ¹H NMR (500 MHz, CDCl₃) δ 6.98 (d, 1H, J=8.3 Hz), 6.83 (d, 1H, J=8.5 Hz), 3.10 (m, 1H), 2.84 (d, 1H, J=18.8 Hz), 2.81-2.57 (m, 2H), 2.46 (m, 1H), 2.32 (m, 1H), 2.03 (m, 3H), 1.86-1.66 (m, 1H), 1.31 (s, 3H), 1.25 (m, 1H), 0.87 (m, 4H), 0.51 (m, 2H), 0.11 (m, 2H); MS (ESI) m/z 317 (M+H)⁺; Anal. Calcd. for C₁₈H₂₄N₂O₃.0.75 H₂O: C, 65.53; H, 7.79; N, 8.49. Found: C, 65.27; H, 7.41; N, 8.23.

For 3: ¹H NMR (500 MHz, CDCl₃) δ 10.36 (s, 1H), 7.80 (s, 1H), 7.03 (s, 1H), 3.16 (m, 1H), 2.95 (d, 1H, J=18.8 Hz), 2.79-2.56 (m, 2H), 2.48 (m, 1H), 2.32 (m, 1H), 1.96 (m, 3H), 1.39 (s, 3H), 1.36 (m, 1H), 0.85 (m, 4H), 0.52 (m, 2H), 0.11 (m, 2H); MS (ESI) m/z 317 (M+H)⁺; Anal. Calcd. for C₁₈H₂₄N₂O₃.0.5 H₂O: C, 66.44; H, 7.74; N, 8.61. Found: C, 66.03; H, 7.33; N, 8.48.

Trifluoromethanesulfonic acid, cis-(±)-3-(cyclopropylmethyl)-1,2,3,4,5,6-hexahydro-6,11-dimethyl-7-nitro-2,6-methano-3-benzazocine-8-yl ester (16). Triethylamine (0.22 g, 1.48 mmol) was added to a solution of 2 (0.47 g, 1.48 mmol) dissolved in 20 mL of CHCl₃. PhN(SO₂CF₃)₂ (0.58 g, 1.63 mmol) was then added and the resulting mixture stirred at 25° C. for 4 h. The solvent was removed on a rotary evaporator and the resulting mixture was purified by gradient silica gel flash chromatography (CH₂Cl₂:CH₃OH; 80:1→40:1) to give 16 (0.59 g, 88%) as an off-white foam. ¹H NMR (500 MHz, CDCl₃) δ 7.30 (d, 1H, J=8.5 Hz), 7.24 (d, 1H, J=8.6 Hz), 3.56 (m, 1H), 3.17 (m, 1H), 3.05 (m, 2H), 2.81 (m, 1H), 2.66 (m, 1H), 2.29-2.04 (m, 2H), 1.90 (m, 1H), 1.34 (m, 4H), 0.87 (m, 4H), 0.69 (m, 2H), 0.28 (m, 2H).

Trifluoromethanesulfonic acid, cis-(±)-3-(cyclopropylmethyl)-1,2,3,4,5,6-hexahydro-6,11-dimethyl-9-nitro-2,6-methano-3-benzazocine-8-yl ester (17). Using a procedure similar to that used to prepare 16, compound 3 was converted to 17 (93%) as off-white foam. ¹H NMR (500 MHz, CDCl₃) δ 7.94 (s, 1H), 7.27 (s, 1H), 3.60 (m, 1H), 3.22-2.94 (m, 3H), 2.84 (m, 1H), 2.68 (m, 1H), 2.30 (m, 1H), 2.11 (m, 2H), 1.41 (s, 3H), 1.38 (m, 1H), 0.84 (m, 4H), 0.69 (m, 2H), 0.29 (m, 2H).

Cis-(±)-3-(cyclopropylmethyl)-1,2,3,4,5,6-hexahydro-6,11-dimethyl-7-nitro-2,6-methano-3-benzazocine-8-carbonitrile (18). To a tube containing 16 (0.27 g, 0.061 mmol) was added under an N₂ blanket, Zn(CN)₂ (0.14 g, 1.22 mmol) and Pd(PPh₃)₄ (0.07 g, 0.061 mmol). DMF (degassed with N₂), 3.0 mL) was then added via a cannula under N₂. The resulting mixture was irradiated with microwaves at 150° C. for 15 min. The resulting mixture was partitioned between water and EtOAc. The organic phase was washed with water (X2) and brine, and then dried over Na₂SO₄, filtered, and concentrated to give a crude product which was purified by silica gel flash chromatography (CH₂Cl₂:CH₃OH:NH₄OH;80:1:0.1) to give 18 (0.14 g, 70%) as an off-white foam. ¹H NMR (500 MHz, CDCl₃) δ 7.52 (d, 1H, J=8.1 Hz), 7.35 (d, 1H, J=8.1 Hz), 3.20 (m, 1H), 3.04 (d, 1H, J=19.1 Hz), 2.86 (m, 1H), 2.68 (m, 2H), 2.47 (m, 1H), 2.34 (m, 1H), 2.10-1.74 (m, 3H), 1.34 (m, 4H), 0.89 (m, 1H), 0.84 (d, 3H, J=7.1 Hz), 0.54 (m, 2H), 0.12 (m, 2H). MS (ESI) m/z 326 (M+H)⁺.

Cis-(±)-3-(cyclopropylmethyl)-1,2,3,4,5,6-hexahydro-6,11-dimethyl-9-nitro-2,6-methano-3-benzazocine-8-carbonitrile (19). Using a procedure similar to that used to prepare 18, compound 17 was converted to 19 (quantitative yield) as an off-white foam. ¹H NMR (500 MHz, CDCl₃) δ 8.05 (s, 1H), 7.75 (s, 1H), 3.22 (m, 1H), 3.08 (d, 1H, J=19.8 Hz), 2.79 (m, 2H), 2.47 (m, 1H), 2.32 (m, 1H), 2.10-1.78 (m, 3H), 1.46 (s, 3H), 1.33 (m, 1H), 0.87 (m, 1H), 0.83 (d, 3H, J=7.1 Hz), 0.54 (m, 2H), 0.12 (m, 2H).

Cis-(±)-3-(cyclopropylmethyl)-1,2,3,4,5,6-hexahydro-6,11-dimethyl-7-nitro-2,6-methano-3-benzazocine-8-carboxamide (4). A solution of 18 (0.11 g, 0.33 mmol) dissolved in t-BuOH (2.0 mL) was heated at 82° C. and KOH (0.056 g, 1.0 mmol) was added. After stirring at 82° C. for 1 h, brine and EtOAc were added. The organic phase was dried over Na₂SO₄, filtered, and concentrated to give a crude product which was purified by silica gel flash chromatography (CH₂Cl₂:CH₃OH:NH₄OH; 20:1:0.1) to give 4 as an off-white solid (0.098 g, 85%). Crystallization of this solid from acetone followed by a recrystallization from i-PrOH/t-BuOH gave crystals having mp 190° C. ¹H NMR (500 MHz, CDCl₃) δ 8.03 (s, 1H), 7.54 (s, 1H), 7.38 (s, 2H), 3.04 (m, 1H), 2.96 (d, 1H, J=19.5 Hz), 2.76 (m, 2H), 2.38 (m, 1H), 2.24 (m, 1H), 2.06-1.56 (m, 3H), 1.19 (m, 4H), 0.79 (m, 1H), 0.73 (d, 3H, J=6.8 Hz), 0.44 (m, 2H), 0.07 (m, 2H); MS (ESI) m/z 344 (M+H)⁺; Anal. Calcd. for C₁₉H₂₅N₃O₃.0.5 H₂O: C, 64.75; H, 7.44; N, 11.92. Found: C, 64.47; H, 7.21; N, 11.56.

Cis-(±)-3-(cyclopropylmethyl)-1,2,3,4,5,6-hexahydro-6,11-dimethyl-9-nitro-2,6-methano-3-benzazocine-8-carboxamide (5). Using a procedure similar to that used to prepare 4, compound 19 was converted to 5 (45%) as an off-white foam. ¹H NMR (500 MHz, CDCl₃) δ 7.80 (s, 1H), 7.42 (s, 1H), 5.89 (m, 2H), 3.20 (m, 1H), 3.03 (d, 1H, J=19.0 Hz), 2.75 (m, 2H), 2.47 (m, 1H), 2.33 (m, 1H), 2.06-1.82 (m, 3H), 1.43 (s, 3H), 1.34 (m, 1H), 0.87 (m, 1H), 0.83 (d, 3H, J=7.1 Hz), 0.53 (m, 2H), 0.12 (m, 2H); MS (ESI) m/z 344 (M+H)⁺; Anal. Calcd. for C₁₉H₂₅N₃O₃.0.25 H₂O: C, 65.59; H, 7.39; N, 12.08. Found: C, 65.39; H, 7.38; N, 11.93.

Cis-(±)-3-(cyclopropylmethyl)-1,2,3,4,5,6-hexahydro-6,11-dimethyl-7-amino-2,6-methano-3-benzazocine-8-carboxamide (6). To a solution of 4 (0.15 g, 0.44 mmol) dissolved in MeOH (20 mL) was added 10% Pd/C (0.093 g). The resulting mixture was subjected to 55 psi H₂ in a Parr shaker for 3 d at 25° C. The mixture was filtered and concentrated to give a crude product that was purified by silica gel flash chromatography (CH₂Cl₂:CH₃OH:NH₄OH; 30:1:0.1) giving 6 (0.060 g, 44%) as a white foam. ¹H NMR (500 MHz, CDCl₃) δ 7.14 (d, 1H, J=8.1 Hz), 6.42 (d, 1H, J=8.1 Hz), 6.12 (s, 1H), 5.58 (br s, 2H), 3.09 (m, 1H), 2.76 (m, 3H), 2.24 (m, 1H), 2.28 (m, 1H), 2.06-1.70 (m, 3H), 1.59 (s, 3H), 1.58 (m, 1H), 0.91 (d, 3H, J=7.1 Hz), 0.86 (m, 1H), 0.51 (m, 2H), 0.10 (m, 2H); MS (ESI) m/z 314 (M+H)⁺; Anal. Calcd. for C₁₉H₂₇N₃O.0.25 H₂O: C, 71.78; H, 8.72; N, 13.22. Found: C, 72.00; H, 8.84; N, 12.98.

Cis-(±)-3-(cyclopropylmethyl)-1,2,3,4,5,6-hexahydro-6,11-dimethyl-9-amino-2,6-methano-3-benzazocine-8-carboxamide (7). Using a procedure similar to that used to prepare 6, compound 5 was converted to 7 (63%) as an off-white foam. ¹H NMR (500 MHz, CDCl₃) δ 7.20 (s, 1H), 6.42 (s, 1H), 5.61 (br s, 2H), 5.42 (s, 2H), 3.10 (m, 1H) 2.84 (d, 1H, J=18.8 Hz), 2.75-2.53 (m, 2H), 2.46 (m, 1H), 2.30 (m, 1H), 2.06-1.80 (m, 3H), 1.35 (s, 3H), 1.27 (m, 1H), 0.87 (m, 1H), 0.84 (d, 3H, J=7.1 Hz), 0.51 (m, 2H), 0.10 (m, 2H); MS (ESI) m/z 314 (M+H)⁺; Anal. Calcd. for C₁₉H₂₇N₃O.0.25 H₂O: C, 71.78; H, 8.72; N, 13.22. Found: C, 72.00; H, 8.73; N, 13.27.

Cis-(±)-7-Amino-3-(cyclopropylmethyl)-1,2,3,4,5,6-hexahydro-6,11-dimethyl-2,6-methano-3-benzazocine-8-carbonitrile (20). A mixture of 6 (0.22 g, 0.70 mmol), POCl₃ (0.11 gm, 0.70 mmol), and pyridine (2.0 mL) was heated at 100° C. for 20 min under microwave radiation and concentrated. The residue was dissolved in 1.0 N HCl and stirred for 1 h at 25° C. The reaction mixture was made basic with saturated Na₂CO₃ and the organic material was extracted into ethyl acetate. The organic layer were washed with brine, dried over Na₂SO₄, filtered and concentrated to give a crude product that was purified by silica gel chromatography (Combiflash—CH₂Cl₂:CH₃OH:NH₄OH) to give 20 as an off-white solid (0.11 g) in 54% yield: ¹H NMR (500 MHz, CDCl₃) δ 7.07 (d, 1H, J=8.5 Hz), 6.41 (d, 1H, J=8.8 Hz), 3.16 (m, 1H), 2.70 (m, 4H), 2.46 (m, 1H), 2.30 (m, 1H), 2.07-1.70 (m, 3H), 1.64 (m, 4H), 0.94 (d, 3H, J=6.8 Hz), 0.88 (m, 1H), 0.53 (m, 2H), 0.12 (m, 2H).

Cis-(±)-9-Amino-3-(cyclopropylmethyl)-1,2,3,4,5,6-hexahydro-6,11-dimethyl-2,6-methano-3-benzazocine-8-carbonitrile (22). A mixture of 19 (0.180 g, 0.55 mmol), 10% Pd/C and CH₃OH (20 mL) was subjected to 40 psi H₂ in a Parr shaker at 25° C. for 15 h. The mixture was filtered and concentrated to give 22 as a crude product that was purified by silica gel chromatography (Combiflash—CH₂Cl₂:CH₃OH:NH₄OH) to give an off-white foam (0.070 g, 47%): ¹H NMR (500 MHz, CDCl₃) δ 7.25 (s, 1H), 6.47 (s, 1H), 4.18 (s, 2H), 3.15 (m, 1H), 2.86 (d, 1H, J=19.0 Hz), 2.80-2.58 (m, 2H), 2.48 (m, 1H), 2.33 (m, 1H), 1.94 (m, 3H), 1.32 (s, 3H), 1.25 (m, 1H), 0.90 (m, 1H), 0.81 (d, 3H, J=7.1 Hz), 0.53 (m, 2H), 0.12 (m, 2H); MS (ESI) m/z 296 (M+H)⁺.

7,8-Fused pyrimidinone derivative 8 and 8,9-Fused pyrimidinone derivative 9. A mixture of 6 (0.035 g, 0.11 mmol) and 2.0 mL of 88% formic acid was heated at 120° C. under microwave radiation for 30 min. The reaction mixture was basified using excess NH₄OH and the organic material was extracted into ethyl acetate. The organic phase was washed with brine, dried over Na₂SO₄ and concentrated giving a crude product that was purified by silica gel chromatography (Combiflash—CH₂Cl₂:CH₃OH:NH₄OH) giving 8 (0.020 gm, 54%). In similar fashion, 7 (0.021 g, 0.067 mmol) was converted to 9 (0.016 gm, 50%). Alternatively, the product from the nitration of cyclazocine containing the 7- and 9-nitro-cyclazocine derivatives 2/3 was converted to a mixture of the 7- and 9-nitro-carbonitrile derivatives 18/19 using the same method as described above for the individual regioisomers. The mixture of 18/19 (1.00 gm, 3.08 mmol) was dissolved in CH₃OH (50 mL) and 10% Pd/C (0.065 g) was added. The resulting mixture was subjected to 20 psi hydrogen in a Parr shaker for 20 h, filtered and concentrated giving a crude product consisting of 6/7 contaminated with 20/22. This crude reaction product (1.01 g) was treated with 10 mL 88% formic acid at 100° C. for 37 h and made basic with excess NaOH/H₂O. The organic materials were extracted into ethyl acetate, washed with brine, dried over Na₂SO₄ and concentrated giving a mixture that was separated by silica gel flash chromatography (hexane:acetone:NH₄OH) to provide 8 (0.298 g) and 9 (0.348 gm) as off-white solids in overall yields (two steps) of 30% and 35%, respectively.

For 8: ¹H NMR (500 MHz, CDCl₃) δ 10.90 (br s, 1H), 8.08 (d, 1H, J=8.1 Hz), 7.99 (s, 1H), 7.25 (d, 1H, J=8.1 Hz), 3.19 (m, 1H), 2.93 (m, 2H), 2.77 (m, 1H), 2.48 (m, 1H), 2.29 (m, 1H), 2.06 (m, 1H), 1.90 (m, 2H), 1.81 (s, 3H), 1.64 (m, 1H), 0.90 (d, 3H, J=7.1 Hz), 0.88 (m, 1H), 0.51 (m, 2H), 0.10 (m, 2H); MS (ESI) m/z 324 (M+H)⁺; Anal. Calcd. for C₂₀H₂₅N₃O: C, 74.27; H, 7.79; N, 12.99. Found: C, 73.95; H, 7.86; N, 12.78.

For 9: ¹H NMR (500 MHz, CDCl₃) δ 11.10 (br s, 1H), 8.19 (s, 1H), 8.05 (s, 1H), 7.48 (s, 1H), 3.23 (m, 1H), 3.14 (d, 1H, J=19.3 Hz), 2.91-2.72 (m, 2H), 2.51 (m, 1H), 2.35 (m, 1H), 2.08-1.86 (m, 3H), 1.52 (s, 3H), 1.38 (m, 1H), 0.90 (m, 1H), 0.87 (d, 3H, J=7.1 Hz), 0.88 (m, 1H), 0.53 (m, 2H), 0.13 (m, 2H); MS (ESI) m/z 324 (M+H)⁺; Anal. Calcd. for C₂₀H₂₅N₃O.0.25 H₂O: C, 73.25; H, 7.84; N, 12.81. Found: C, 73.14; H, 7.90; N, 12.38.

7,8-Fused aminopyrimidine derivative 10. A mixture of 20 (0.11 g, 0.38 mmol), CH(OCH₃)₃ (2 mL) and 4 Å molecular sieves was heated at 140° C. for 48 h. The reaction mixture was filtered and concentrated to give imidate intermediate 21 (0.120 g) which, without further purification, was combined with methanol saturated with ammonia gas. The resulting mixture was heated for 1 h at 100° C. under microwave radiation and then made basic with concentrated ammonia. After dilution with H₂O, the organic material was extracted into CH₂Cl₂ and the organic layer was washed with brine, dried over Na₂SO₄ and concentrated to give mixture that was purified by silica gel chromatography (Combiflash—CH₂Cl₂:CH₃OH:NH₄OH) and crystallization. The desired product 10 (0.074 gm) was obtained in 56% yield (2 steps) as an off-white solid: mp 190° C.: NMR (500 MHz, CDCl₃) δ 8.56 (s, 1H), 7.49 (d, 1H, J=8.3 Hz), 7.20 (d, 1H, J=8.3 Hz), 5.54 (s, 2H), 3.20 (m, 1H), 2.92 (m, 2H), 2.76 (m, 1H), 2.48 (m, 1H), 2.29 (m, 1H), 2.19 (m, 1H), 1.94 (m, 4H), 1.89 (s, 3H), 0.91 (d, 3H, J=7.1 Hz), 0.89 (m, 1H), 0.51 (m, 2H), 0.10 (m, 2H); MS (ESI) m/z 323 (M+H)⁺; Anal. Calcd. for C₂₀H₂₆N₄.0.25 H₂O: C, 73.47; H, 8.17; N, 17.14. Found: C, 73.59; H, 8.04; N, 16.92.

7,8-Fused benzylaminopyrimidine derivative 12. Using a procedure similar to that used to prepare 10, compound 21 was treated with benzylamine to provide 12 (69%) as an off-white foam: NMR (500 MHz, CDCl₃) δ 8.64 (s, 1H), 7.44 (d, 1H, J=8.5 Hz), 7.40-7.30 (m, 5H); 7.15 (d, 1H, J=8.3 Hz), 5.81 (m, 1H), 4.83 (d, 2H, J=5.4 Hz), 3.20 (m, 1H), 2.91 (m, 2H), 2.77 (m, 1H), 2.48 (m, 1H), 2.29 (m, 1H), 2.22 (m, 1H), 1.93 (m, 2H), 1.90 (s, 3H), 1.88 (m, 1H); 0.90 (d, 3H, J=7.1 Hz), 0.88 (m, 1H), 0.51 (m, 2H), 0.10 (m, 2H); MS (ESI) m/z 413 (M+H)⁺; Anal. Calcd. for C₂₇H₃₂N₄.0.5 H₂O: C, 76.92; H, 7.89; N, 13.29. Found: C, 76.77; H, 7.99; N, 12.90.

7,8-Fused biphenylethylaminopyrimidine derivative 14. Using a procedure similar to that used to prepare 10, compound 21 was treated with 4-biphenylethylamine to provide to 14 (71%) as an off-white foam: NMR (500 MHz, CDCl₃) δ 8.64 (s, 1H), 7.58 (m, 4H), 7.45 (m, 2H), 7.34 (m, 3H), 7.29 (d, 1H, J=8.5 Hz), 7.13 (d, 1H, J=8.5 Hz), 5.56 (m, 1H), 3.93 (m, 2H), 3.19 (m, 1H), 3.06 (t, 2H, J=6.6 Hz), 2.89 (m, 2H), 2.77 (m, 1H), 2.47 (m, 1H), 2.28 (m, 1H), 2.21 (m, 1H), 1.90 (s, 3H), 1.87 (m, 1H); 1.63 (m, 2H), 0.90 (d, 3H, J=7.1 Hz), 0.88 (m, 1H), 0.51 (m, 2H), 0.10 (m, 2H); MS (ESI) m/z 503 (M+H)⁺; Anal. Calcd. for C₃₄H₃₈N₄.0.5 H₂O: C, 79.81; H, 7.68; N, 10.95. Found: C, 79.88; H, 7.66; N, 10.83.

8,9-Fused aminopyrimidine derivative 11. Using a procedure similar to that used to prepare 10, compound 22 was converted to imidate intermediate 23 which was then converted to 11 (86%) as an off-white foam: ¹H NMR (500 MHz, CDCl₃) δ 8.56 (s, 1H), 7.62 (s, 1H), 7.58 (s, 1H), 6.00 (s, 2H), 3.23 (m, 1H), 3.18 (d, 1H, J=19.0 Hz), 2.89 (m, 1H), 2.73 (m, 1H), 2.51 (m, 1H), 2.35 (m, 1H), 2.01 (m, 3H), 1.48 (s, 3H), 1.35 (m, 1H), 0.89 (m, 1H), 0.87 (d, 3H, J=7.3 Hz), 0.53 (m, 2H), 0.13 (m, 2H); MS (ESI) m/z 323 (M+H)⁺; C₂₀H₂₆N₄.0.25 H₂O: C, 73.47; H, 8.17; N, 17.14. Found: C, 73.33; H, 8.03; N, 16.85.

8,9-Fused benzylaminopyrimidine derivative 13. A mixture of 7 (0.084 g, 0.27 mmol), POCl₃ (0.41 g, 2.7 mmol), and DMF (3.0 mL) was heated at 100° C. under microwave radiation for 10 min and concentrated. The resulting dark oil was dissolved in H₂O, made basic with Na₂CO₃ and extracted (X3) with CH₂Cl₂. The combined organic extracts were dried over Na₂SO₄ and concentrated to give mixture that was purified by silica gel chromatography (CH₂Cl₂:CH₃OH:NH₄OH) giving the desired amidine intermediate 24 in 89% yield. Treatment of 24 (0.12 g, 0.34 mmol) with benzylamine (0.044 g, 0.41 mmol) and excess 30% HOAc in CH₃CN at 160° C. under microwave radiation for 20 min provided, after concentration, an oil that was partitioned between saturated Na₂CO₃ and CH₂Cl₂. The organic phase was washed with brine, dried over Na₂SO₄ and concentrated to give a crude product that was purified by silica gel chromatography (Combiflash—CH₂Cl₂:CH₃OH:NH₄OH) giving 13 (0.16 g) 92% yield as an off-white solid: NMR (500 MHz, CDCl₃) δ 8.64 (s, 1H), 7.56 (m, 1H), 7.50 (s, 1H), 7.44 (d, 2H, J=7.3 Hz), 7.39 (t, 2H, J=7.3 Hz), 7.34 (d, 1H, J=7.3 Hz), 5.94 (br s, 1H), 4.90 (m, 2H), 3.22 (m, 1H), 3.17 (d, 1H, J=19.0 Hz), 2.88 (m, 1H), 2.72 (m, 1H), 2.51 (m, 1H), 2.34 (m, 1H), 1.99 (m, 3H), 1.47 (s, 3H), 1.33 (m, 1H), 0.88 (m, 1H), 0.86 (d, 3H, J=7.1 Hz), 0.52 (m, 2H), 0.12 (m, 2H); MS (ESI) m/z 413 (M+H)⁺; C₂₇H₃₂N₄.H₂O: C, 75.31; H, 7.96; N, 13.01. Found: C, 75.64; H, 7.73; N, 13.02.

8,9-Fused biphenylethylaminopyrimidine derivative 15. Using a procedure similar to that used to prepare 13, compound 24 was treated with 4-biphenylethylamine to provide to 15 (86%) as an off-white foam: NMR (500 MHz, CDCl₃) δ 8.63 (s, 1H), 7.58 (m, 4H), 7.54 (s, 1H), 7.45 (m, 2H), 7.36 (m, 4H), 5.72 (m, 1H), 3.96 (m, 2H), 3.20 (m, 1H), 3.16 (d, 1H, J=19.1 Hz), 3.09 (t, 2H, J=7.1 Hz), 2.80 (m, 1H), 2.71 (m, 1H), 2.50 (m, 1H), 2.34 (m, 1H), 2.04-1.94 (m, 3H), 1.43 (s, 3H), 1.30 (m, 1H), 0.88 (m, 1H), 0.85 (d, 3H, J=7.1 Hz), 0.52 (m, 2H), 0.12 (m, 2H); MS (ESI) m/z 503 (M+H)⁺; Anal. Calcd. for C₃₄H₃₈N₄.0.5 H₂O: C, 79.81; H, 7.68; N, 10.95. Found: C, 79.52; H, 7.64; N, 10.83.

Cis-(±)-3-(cyclopropylmethyl)-N-(diphenylmethylene)-1,2,3,4,5,6-hexahydro-6,11-dimethyl-7-nitro-2,6-methano-3-benzazocine-8-amine 25. To a tube containing benzophenoneimine (0.10 g, 0.56 mmol), Pd(OAc)₂ (0.010 g, 0.045 mmol), BINAP (0.014 g, 0.022 mmol), and Cs₂CO₃ (0.18 g, 0.56 mmol) was added 16 (0.20 g, 0.45 mmol) dissolved in 5 mL toluene. The reaction mixture was heated at 150° C. for 15 min under microwave radiation. Upon cooling to 25° C., the mixture was diluted with EtOAc, filtered and concentrated in vacuo. The resulting residue was purified by silica gel flash chromatography giving 25 (0.12 g, 56%) as an off-white solid. ¹H NMR (500 MHz, CDCl₃) δ 7.73-7.28 (m, 10H), 6.78 (d, 1H, J=8.7 Hz), 6.18 (d, 1H, J=8.7 Hz), 3.05 (m, 1H), 2.80 (m, 1H), 2.63 (m, 1H), 2.57 (m, 1H), 2.44 (m, 1H), 2.30 (m, 1H), 1.82 (m, 2H), 1.35 (s, 3H), 1.25 (m, 1H), 0.84 (d, 3H, J=7.0 Hz), 0.51 (m, 2H), 0.10 (m, 2H). MS (ESI) m/z 480 [(M+H)⁺].

Cis-(±)-3-(cyclopropylmethyl)-N-(diphenylmethylene)-1,2,3,4,5,6-hexahydro-6,11-dimethyl-9-nitro-2,6-methano-3-benzazocine-8-amine 26. Using a procedure similar to that used to prepare 25, compound 17 was converted to 26 (88%) as an off-white foam. MS (ESI) m/z 480 [(M+H)⁺].

Cis-(±)-3-(cyclopropylmethyl)-1,2,3,4,5,6-hexahydro-6,11-dimethyl-7-nitro-2,6-methano-3-benzazocine-8-amine 27. Compound 25 (0.13 g, 0.26 mmol) was dissolved in 2 mL THF and 4 mL of 3N HCl was added. The reaction mixture was stirred at 25° C. for 30 min and was made basic through the addition of conc. NH₄OH. The mixture was treated with ethyl acetate and the organic phase was dried over Na₂SO₄, filtered, and concentrated to give a crude solid produce which was purified by silica gel flash chromatography to give 27 (0.080 g, 98%) as an off-white solid. ¹H NMR (500 MHz, CDCl₃) δ 6.98 (d, 1H, J=8.1 Hz), 6.63 (d, 1H, J=8.1 Hz), 3.93 (s, 2H), 3.10 (m, 1H), 2.80 (m, 2H), 2.66 (m, 1H), 2.60 (m, 1H), 2.48 (m, 1H), 2.43 (m, 1H), 2.36 (s, 1H), 2.32 (m, 1H), 2.00 (m, 1H), 1.82 (m, 2H), 1.30 (s, 3H), 0.82 (d, 3H, J=7.2 Hz), 0.52 (m, 2H), 0.11 (m, 2H). MS (ESI) m/z 316 [(M+H)⁺].

Cis-(±)-3-(cyclopropylmethyl)-1,2,3,4,5,6-hexahydro-6,11-dimethyl-9-nitro-2,6-methano-3-benzazocine-8-amine 28. Using a procedure similar to that used to prepare 27, compound 26 was converted to 28 (88%) as an off-white foam. ¹H NMR (500 MHz, CDCl₃) δ 7.81 (s, 1H), 6.67 (s, 1H), 5.88 (s, 2H), 3.25 (m, 1H), 2.87 (d, 1H, J=18.0 Hz), 2.70 (m, 2H), 2.48 (dd, 1H, J=5.0 Hz), 2.33 (dd, 1H, J=5.0 Hz), 2.04 (m, 1H), 1.92 (m, 1H), 1.31 (s, 3H), 1.25 (m, 1H), 0.83 (d, 3H, J=8.1 Hz), 0.50 (m, 2H), 0.09 (m, 2H). MS (ESI) m/z 316 [(M+H)⁺].

7,8-Fused triazole derivative 29. Compound 27 (0.060 g), 10% Pd/C (0.006 gm) and methanol (2 mL) was subjected to 42 psi H₂ in a Parr shaker for 8 h at 25° C. The mixture was filtered and the filtrate concentrated giving a somewhat unstable diamine product (0.056 gm, 100%). A portion (0.025 gm) of this crude product was dissolved in 1.0 mL HOAc. To this solution was added NaNO₂ (0.006 g) and the resulting mixture stirred for 1 h at 25° C. The mixture was basified with excess conc. NH₄OH and the organic material was extracted into ethyl acetate. The extracts were dried over Na₂SO₄, filtered, and concentrated to give a crude solid produce which was purified by silica gel flash chromatography to give 29 (0.016 g, 62%) as an off-white foam. ¹H NMR (500 MHz, CDCl₃) δ 10.68 (br, 1H), 7.63 (d, 1H, J=8.4 Hz), 7.17 (d, 1H, J=8.4 Hz), 3.30 (s, 1H), 3.10 (d, 1H, J=18.6 Hz), 2.92 (m, 1H), 2.86 (m, 1H), 2.60 (m, 1H), 2.43 (m, 1H), 2.11 (m, 2H), 2.03 (m, 2H), 1.91 (s, 3H), 1.79 (d, 1H, J=8.1 Hz), 0.94 (d, 3H, J=7.2 Hz),0.52 (m, 2H), 0.13 (m, 2H). MS (ESI) m/z 296 [(M+H)]⁺. Anal. Calcd. for C₁₈H₂₄N₄.0.33 H₂O: C, 71.48; H, 8.14; N, 18.14. Found: C,71.48; H,8.24; N, 18.53.

8,9-Fused triazole derivative 30. Using a procedure similar to that used to prepare 29, compound 28 was converted to 30 (68% overall yield) as an off-white foam.

¹H NMR (500 MHz, CDCl₃) δ 8.40-8.80 (m, 1H), 7.82 (d, 1H, J=3.0 Hz), 7.58 (s, 1H), 3.23 (d, 1H, J=18.0 Hz), 2.88 (d, 1H, J=18.5 Hz), 2.84 (d, 1H, J=12.0 Hz), 2.62 (m, 1H), 2.49 (m, 1H), 2.10 (s, 3H), 2.04 (m, 2H), 1.48 (s, 3H), 1.39 (d, 1H, J=12.5 Hz), 0.93 (m, 1H), 0.89 (d, 3H, J=7.0 Hz), 0.52 (d, 2H, J=7.5 Hz), 0.14 (m, 2H). MS (ESI) m/z 296 [(M+H)⁺]. Anal. Calcd. for C₁₈H₂₄N₄.0.70 H₂O: C, 70.00; H, 8.30; N, 18.13. Found: C, 70.44; H, 8.10; N, 17.78.

Cis-(±)-3-(cyclopropylmethyl)-N-(phenylmethyl)-1,2,3,4,5,6-hexahydro-6,11-dimethyl-9-nitro-2,6-methano-3-benzazocine-8-amine 32. Benzylamine (0.18 mL, 1.65 mmol) was added to a flask containing the solution of 17 (0.248 gm, 0.55 mmol) in 5 mL CH₃CN, under argon at room temperature. A reflux condenser was attached and the reaction mixture was stirred at reflux for 18 hours. TLC showed the completion of reaction. The cooled reaction mixture was diluted with methylene chloride and washed with 0.1M NaOH solution and brine. The combined organic layer was dried over sodium sulfate and concentrated to give orange colored oil, which was purified by flash chromatography (CH₂Cl₂:CH₃OH:NH₄OH; 10:1:0.1) to give 32 as an orange foam (0.171 gm, 77%); mp 215-216° C. ¹H NMR (500MHz, CDCl₃) δ 8.23 (t, 1H), 7.88 (s, 1H), 7.36 (m, 4H), 7.29 (m, 1H), 6.65 (s, 1H), 3.12-3.10 (m, 1H), 2.90 (d, 1H, J=18.5 Hz), 2.70-2.67 (m, 1H), 2.61 (d, 0.5H, J=1 Hz), 2.60 (d, 0.5H, J=1 Hz), 2.48-2.44 (m, 1H), 2.31-2.28 (m, 1H), 2.01-1.96 (m, 1H), 1.90-1.83 (m, 2H), 1.22-1.20 (m, 3H), 1.19-1.18 (m, 1H), 0.83 (m, 1H), 0.82 (s, 1.5H), 0.81 (s, 1.5H), 0.51-0.49 (m, 2H), 0.11-0.09 (m, 2H) ppm. MS (ESI) m/z 406 [(M+H)⁺].

Cis-(±)-3-(cyclopropylmethyl)-1,2,3,4,5,6-hexahydro-6,11-dimethyl-2,6-methano-3-benzazocine-8,9-diamine 33. 15 mL Methanol was added to the reaction flask containing compound 32 (0.17 gm, 0.42 mmol), ammonium formate (0.264 gm, 4.20 mmol) and 10% Pd/C (0.046 gm, 0.042 mmol). The reaction mixture was then stirred at reflux for 20 hours. After the completion of reaction, the mixture was filtered through celite and the residue was washed with excess methanol and the combined methanol layer was concentrated. This concentrated matter was then partitioned between methylene chloride and saturated sodium bicarbonate. The combined organic layer was dried over sodium sulfate, filtered, and concentrated to give crude solid produce which was purified by silica gel flash chromatography (CH₂Cl₂:CH₃OH:NH₄OH; 10:1:0.1) to give the somewhat unstable diamino compound 33 (0.080 gm, 67%) as an off-white foam. ¹H NMR (500 MHz, CDCl₃) δ 6.57 (s, 1H), 6.41 (s, 1H), 3.28 (s, 4H), 3.09-3.07 (m, 1H), 2.78-2.74 (d, 1H, J=18 Hz), 2.74-268 (m, 0.5H), 2.67-2.66 (m, 0.5H), 2.56-2.55 (d, 0.5H, J=6 Hz), 2.52-2.51 (d, 0.5H, J=5.5 Hz), 2.48-2.44 (m, 1H), 2.31-2.28 (m, 1H), 1.86-1.78 (m, 2H), 1.29 (m, 4H), 0.87-0.83 (m, 4H), 0.50-0.48 (m, 2H), 0.10-0.08 (m, 2H) ppm. HRMS m/z Calcd, 286.2283; Found, 286.2264 for C₁₈H₂₇N₃.

8,9-Fused imidazole derivative 34. A solution of compound 33 (0.240 gm, 0.84 mmol) in 10 mL formic acid was stirred at refluxed for 20 hours under argon. After the completion of the reaction, it was cooled to 0° C. and carefully basified with conc. NH₄OH. The organic matter was then extracted into methylene chloride. The extracts were dried over sodium sulfate, filtered, and concentrated to give crude solid produce which was purified by silica gel flash chromatography (CH₂Cl₂:CH₃OH:NH₄OH; 40:9:1) to give compound 34 (0.170 gm, 69%) as an off-white foam. This product was converted to its HCl salt by treatment with 1M HCl in Et₂O. The crude salt was crystallized with EtOAC/MeOH to give white crystalline solid having mp 218° C. ¹H NMR (500 MHz, CD₃OD) δ 9.32 (s, 1H), 7.85 (s, 1H), 4.89 (s, 1H), 4.04 (s, 1H), 3.52-3.49 (m, 1H), 3.42 (m, 1H), 3.35 (m, 1H), 3.12-3.08 (m, 1H), 2.60-2.58 (m, 1H), 2.37 (m, 1H), 2.21 (m, 1H), 1.73 (m, 1H), 1.62 (s, 3H), 1.22 (m, 1H), 1.00 (d, 3H, J=7 Hz), 0.94 (m, 1H), 0.80-0.77 (m, 2H), 0.50 (m, 2H) ppm. MS (ESI) m/z 296 [(M+H)⁺]. Anal. Calcd. for C₁₉H₂₅N₃.2HCl.0.5H₂O: C, 60.48; H, 7.48; N, 11.14. Found C, 60.14; H, 7.63; N, 10.84.

8,9-Fused imidazole derivative 35. A solution of compound 33 (0.090 gm, 0.32 mmol) in 1.5 mL acetic acid was heated under microwave radiation at 130° C. for 30 min. After the completion of the reaction, it was cooled to 0° C. and carefully basified with conc. NH₄OH. The organic matter was then extracted into methylene chloride. The extracts were dried over sodium sulfate, filtered, and concentrated to give crude solid produce which was purified by silica gel flash chromatography (CH₂Cl₂:CH₃OH:NH₄OH; 10:1:0.1) to give the compound 35 (0.061 gm, 62%) as an off-white foam. ¹H NMR (500 MHz, CDCl₃) δ 10.40 (s, br, 1H), 6.56 (s, 1H), 6.41 (s, 1H), 3.28 (s, 3H), 3.11-3.09 (m, 1H), 2.78-2.74 (d, 1H, J=18 Hz), 2.71-2.68 (m, 1H), 2.58-2.53 (m, 1H), 2.49-2.46 (m, 1H), 2.33-2.29 (m, 1H), 2.08-2.03 (m, 1H), 1.88-1.79 (m, 2H), 1.29 (s, 3H), 1.27-1.23 (m, 1H), 0.87 (m, 1H), 0.85 (d, 3H, J=7 Hz), 0.50-0.48 (m, 2H), 0.11-0.09 (m, 2H), ppm. MS (ESI) m/z 310 [(M+H)]⁺. Anal. Calcd. for C₂₀H₂₇N₃.0.63H₂O: C, 74.67; H, 9.16; N, 13.10. Found C, 74.90; H, 8.90; N, 13.10.

8,9-Fused imidazole derivative 36. 2 mL of phosgene solution in toluene was added to a THF solution of compound 33 (0.075 gm, 0.26 mmol) and the reaction was stirred for 16 hours at room temperature. The reaction mixture was diluted with water and basified with conc. NH₄OH. The organic matter was then extracted into methylene chloride. The extracts were dried over sodium sulfate, filtered, and concentrated to give crude solid produce which was purified by silica gel flash chromatography (CH₂Cl₂:CH₃OH:NH₄OH; 10:1:0.1) to give the compound 36 (0.064 gm, 78%) as an off-white solid, which was crystallized with EtOAc/MeOH to give a solid having mp of 172° C. ¹H NMR (500 MHz, CDCl₃) δ 8.63 (s, 1H), 8.52 (s, 1H), 6.93 (s, 1H), 6.75 (s, 1H), 3.12 (s, 1H), 2.95 (d, 1H, J=18 Hz), 2.74-2.69 (m, 2H), 2.50-2.46 (m, 1H), 2.33-2.29 (m, 1H), 1.98-187 (m, 3H), 1.37 (s, 3H), 1.29 (m, 1H), 0.86 (d, 3H, J=7 Hz), 0.51 (m, 2H), 0.11 (m, 2H) ppm. MS (ESI) m/z 312 [(M+H)⁻]. Anal. Calcd. for C₁₉H₂₅N₃O.0.25H₂O: C, 73.28; H, 8.09; N, 13.49. Found C, 72.23; H, 8.14; N, 13.30.

8,9-Fused imidazole derivative 37. Using a procedure similar to that used to prepare 35, compound 33 was treated with CH₃CH₂CO₂H to provide 37 (88% yield) as an off-white foam. ¹H NMR (500 MHz, CDCl₃) δ 9.04 (s, br, 1H), 7.62 (s, 1H), 7.06 (s, 1H), 3.17 (s, br, 1H), 3.12-3.04 (m, 1H), 2.94-2.80 (m, 3H), 2.69 (d, 1H, J=12 Hz), 2.53-2.48 (m, 1H), 2.37-2.30 (m, 1H), 2.05-1.78 (m, 3H), 1.73 (s, br, 2H), 1.43 (t, 3H, J=9 Hz), 1.37-1.23 (m, 3H), 0.87 (d, 3H, J=7 Hz), 0.54-0.48 (m, 2H), 0.19-0.08 (m, 2H) ppm.

8,9-Fused imidazole derivative 38. Using a procedure similar to that used to prepare 35, compound 33 was treated with (CH₃)₂CHCO₂H to provide 38 (71% yield) as an off-white foam. ¹H NMR (500 MHz, CDCl₃) δ 9.79 (s, br, 1H), 7.64 (s, 1H), 7.08 (s, 1H), 3.18 (s, br, 2H), 3.07 (d, 1H, J=18 Hz), 2.87 (dd, 1H, J₁_{1,2}=6, 19 Hz), 2.71 (dd, 1H, J_{1,2}=2, 12 Hz), 2.54-2.48 (m, 1H), 2.39-2.32 (m, 1H), 2.07-1.85 (m, 3H), 1.44 (d, 6H, J=7 Hz), 1.42-1.15 (m, 5H), 0.87 (d, 3H, J=7 Hz), 0.56-0.48 (m, 2H), 0.13-0.08 (m, 2H) ppm.

8,9-Fused imidazole derivative 39. Using a procedure similar to that used to prepare 35, compound 33 was treated with c-C₃H₅CO₂H to provide 39 (86% yield) as a yellow foam. ¹H NMR (500 MHz, CDCl₃) δ 8.95 (s, br, 1H), 7.57 (s, 1H), 7.03 (s, 1H), 3.17 (s, br, 1H), 3.06 (dd, 1H, J_{1,2}=8, 18 Hz), 2.90-2.80 (m, 1H), 2.73-2.65(m, 1H), 2.55-2.46 (m, 1H), 2.40-2.30 (m, 1H), 2.10-1.80 (m, 4H), 1.75-1.60 (m, 2H), 1.47-1.40 (m, 2H), 1.39-1.23 (m, 2H), 1.20-1.13 (m, 2H), 1.10-1.05 (m, 1 H), 0.95-0.8 (m, 3H), 0.56-0.47 (m, 2H), 0.19-0.07 (m, 2H) ppm.

Cis-(±)-3-(cyclopropylmethyl)-1,2,3,4,5,6-hexahydro-6,11-dimethyl-2,6-methano-3-benzazocine-7,8-diamine 40. To a solution of compound 28 (0.080 gm, 0.25 mmol) in 10 mL of methanol was added 10% Pd/C (0.008 gm). The suspension was placed in a Parr hydrogenation apparatus and shaken for 8 hours at 25° C. at a pressure of 40 psi. The reaction mixture was then filtered over Celite. The filtrate was concentrated in vacuo, to give compound 40 as a somewhat unstable off-white foam in quantitative yield. ¹H NMR (500 MHz, CDCl₃) δ 6.60 (d, 1H, J=8 Hz), 6.46 (d, 1H, J=8 Hz), 5.00-4.00 (s, br, 2H), 3.46 (s, 2H), 3.09 (d, 1H, J=11 Hz), 2.99 (dd, 1H, J_{1,2}=6, 14 Hz), 2.80 (d, 1H, J=14 Hz), 2.73-2.68 (m, 1H), 2.64-2.59 (m, 1H), 2.27 (t, 1H, J=11 Hz), 2.14 (d, 1H, J=5 Hz), 2.02 (s, 2H), 1.80 (d, 1H, J=13 Hz), 1.62 (s, 3H), 1.01-1.00 (m, 1H), 0.96 (d, 3H, J=7 Hz), 0.61 (d, 2H, J=8 Hz), 0.25 (dd, 2H, J_{1,2}=4, 13 Hz) ppm.

7,8-Fused imidazole derivative 41. Using a procedure similar to that used to prepare 35, compound 40 was treated with HCO₂H to provide 41 (80% yield) as an off-white foam. ¹H NMR (500 MHz, CDCl₃) δ 9.18 and 9.02 (s, br, 1H), 7.97 and 7.95 (s, 1H), 7.58 and 7.27 (d, 1H, J=9 Hz), 7.04 and 7.02 (1H, d, J=9 Hz), 3.19-3.17 (m, 1H), 3.03-2.95 (d, 1H, J=18 Hz), 2.88-2.81 (m, 1H), 2.73-2.69 (m, 1H), 2.52-2.48 (dd, 1H, J=7, 13 Hz), 2.35-2.28 (dd, 1H, J=6, 13 Hz), 2.02-1.88 (m, 3H), 1.94 and 1.69 (s, 3H), 1.60-1.59 (m, 1H), 0.96 and 0.93 (d, 3H, J=7 Hz), 0.89-0.86 (m, 1H), 0.52-0.50 (m, 2H), 0.13-0.10 (m, 2H) ppm.; MS (ESI) m/z 296 [(M+H)⁻]; Anal. Calcd. for C₁₉H₂₅N₃.0.5H₂O.0.4CH₂Cl₂: C, 68.86; H, 7.98; N, 12.42. Found C, 69.19; H, 7.53; N, 11.98.

7,8-Fused imidazole derivative 42. Using a procedure similar to that used to prepare 35, compound 40 was treated with CH₃CO₂H to provide 42 (97% yield) as an off-white foam. ¹H NMR (500 MHz, CDCl₃) δ 8.80 (s, br, 1H), 7.44 (d, 1H, J=9 Hz), 6.97 (d, 1H, J=9 Hz), 3.20 (s, br, 1H), 3.02-2.94 (m, 1H), 2.90-2.70 (m, 3H), 2.55-2.48(m, 1H), 2.05-1.82 (m, 4H), 1.66 (s, 3H), 1.60-1.55 (m, 1H), 0.93 (d, 3H, J=7 Hz), 0.92-0.83 (m, 1H), 0.56-0.47 (m, 2H), 0.18-0.08 (m, 2H) ppm. Anal. Calcd. for C₂₀H₂₇IN₃ 0.5 H₂O: C, 75.43; H, 8.86; N, 13.19. Found C, 74.99; H, 8.51; N, 12.98.

7,8-Fused isoxazole derivative 43. To a solution of compound 18 (0.48 gm, 1.50 mmol) in 10 mL of HCl (37%) was added SnCl₂.2H₂O (1.67 gm, 7.50 mmol). The reaction mixture was stirred overnight at 25° C. and then carefully basified with 2 N NaOH solution. The organic matter was then extracted into EtOAc. The extracts were dried over sodium sulfate, filtered, and concentrated to give crude solid produce which was purified by silica gel flash chromatography (CH₂Cl₂:CH₃OH:NH₄OH; 10:1:0.1) to give compound 43 (0.18 gm, 39%) as an off-white foam. ¹H NMR (500 MHz, CDCl₃) δ 6.98 (d, 1H, J=9 Hz), 6.44 (d, 1H, J=9 Hz), 5.04 (s, 2H), 3.14-3.11 (m, 1H), 2.78-2.57 (m, 3 H), 2.48-2.43 (m, 1H), 2.30-2.26 (m, 1H), 2.03-1.96 (m, 1H), 1.94-1.88 (m, 1H), 1.86-1.80 (m, 1H), 1.80-1.72 (m, 1H), 1.67 (s, 3H), 0.94 (d, 3H, J=7 Hz), 0.91-0.82 (m, 1H), 0.56-0.46 (m, 2H), 0.14-0.06 (m, 2H) ppm. MS (ESI) m/z 312 [(M+H)⁺]. Anal. Calcd. for C₁₉H₂₅N₃O.0.1 H₂O: C, 72.86; H, 8.11; N, 13.42. Found C, 72.62; H, 8.20; N, 13.19.

In general, the chemistry described above works in the presence of the variety of functional groups found on known core structures. The exceptions would be morphine and congeners having a free 6-OH, which can be protected by a TBDPS (t-butyldiphenylsilyl) group [see Wentland et al., “Selective Protection and Functionalization of Morphine . . . ”, J. Med. Chem. 43, 3558-3565 (2000)], the entire contents of which are incorporated herein by reference.

I claim:

1. A compound of formula: 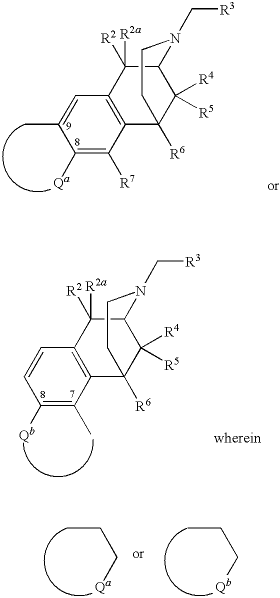
is a five- or six-membered heterocyclic ring, which may be substituted or further fused to form a residue of one to three rings;
Q^{a} is chosen from 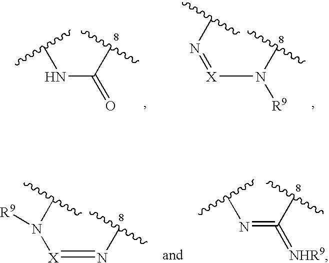
with the proviso that, when Q^{a} is 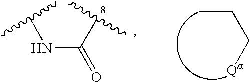
is not a pyridinone ring;
Q^{b} is chosen from 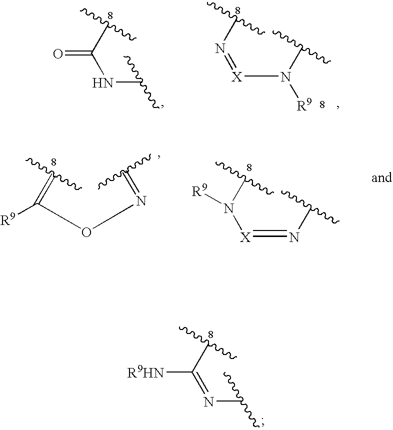
X is N or CR⁹;
R² and R^{2a} are both hydrogen or taken together R² and R^{2a} are ═O;
R³ is chosen from hydrogen, (C₁-C₈)hydrocarbon, heterocyclyl, heterocyclylalkyl and hydroxyalkyl;
R⁴ is chosen from hydrogen, hydroxy, amino, (C₁-C₆)alkoxy, (C₁-C₂₀)alkyl and (C₁-C₂₀)alkyl substituted with hydroxy or carbonyl;
R⁵ is (C₁-C₆)alkyl;
R⁶ is (C₁-C₆)alkyl;
R⁷ is chosen from hydrogen, NHR⁹ and hydroxy; or together R⁴, R⁵, R⁶ and R⁷ may form from one to three rings, said rings having optional additional substitution;
R⁹ in each of its occurrences is independently chosen from H, alkyl and 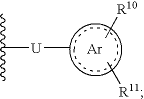
U is (CH₂)ₙ, wherein one or more CH₂ may be replaced by —O—, cycloalkyl or —CR^{1a}R^{1b};
R^{1a} and R^{1b} are chosen independently from hydrogen, halogen, (C₁-C₆)alkyl, (C₁-C₆)alkoxy and (C₁-C₆)alkylthio;
Ar is an aryl or heteroaryl residue of one to three rings;
R¹⁰ is one or two residues chosen independently from hydrogen, hydroxyl, halogen, (C₁-C₆)alkyl, (C₁-C₆)alkoxy, halo(C₁-C₆)alkyl and halo(C₁-C₆)alkoxy and (C₁-C₆)alkylthio;
R¹¹ is H or 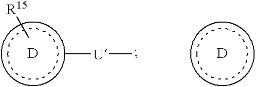
is an aryl or heteroaryl residue of one to three rings;
U′ is (CH₂)ₘ, wherein one or more CH₂ may be replaced by —O—, cycloalkyl, —CR^{1a}R^{1b}, —C(═O)— or —NH—;
R¹⁵ is one or two residues chosen independently from hydrogen, hydroxyl, halogen, (C₁-C₆)alkyl, (C₁-C₆)alkoxy, halo(C₁-C₆)alkyl and halo(C₁-C₆)alkoxy and (C₁-C₆)alkylthio;
m is zero or an integer from 1 to 6; and
n is an integer from 1 to 6.

2. A 2,6-methano-3-benzazocine according to claim 1 of formula 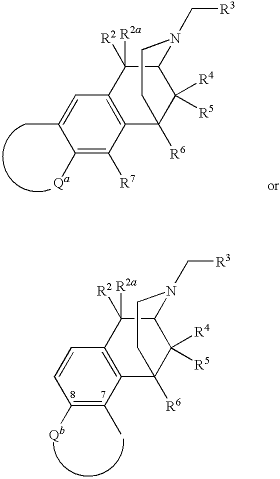
wherein:
R³ is chosen from hydrogen, (C₁-C₇)hydrocarbon, heterocyclyl, and hydroxyalkyl;
R⁴ is chosen from hydrogen, hydroxy, (C₁-C₆)alkoxy, (C₁-C₂₀)alkyl and (C₁-C₂₀)alkyl substituted with hydroxy or carbonyl;
R⁵ is (C₁-C₆)alkyl;
R⁶ is (C₁-C₆)alkyl; and
R⁷ is hydrogen or hydroxy.

3. A 2,6-methano-3-benzazocine according to claim 2 wherein:
R³ is chosen from hydrogen, cyclopropyl, cyclobutyl, phenyl, vinyl, dimethylvinyl, hydroxycyclopropyl, furanyl and tetrahydrofuranyl;
R⁴ is hydrogen;
R⁵ is methyl;
R⁶ is methyl or ethyl; and
R⁹ is chosen from hydrogen, methyl, benzyl and 2-(biphenylyl)ethyl.

4. A morphinan according to claim 1 wherein together R⁵ and R⁶ form one ring, said morphinan having the structure: 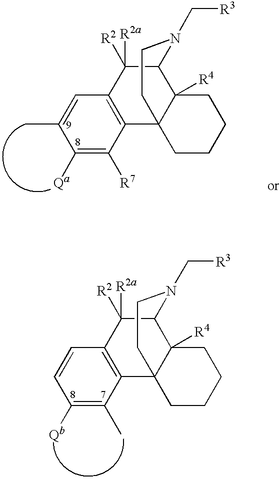
wherein:
R³ is chosen from hydrogen, (C₁-C₇)hydrocarbon, heterocyclyl, and hydroxyalkyl.

5. A morphinan according to claim 4 wherein
R² and R^{2a} are hydrogen;
R³ is chosen from hydrogen, cyclopropyl, cyclobutyl, vinyl and tetrahydrofuranyl;
R⁴ is hydrogen, hydroxy or amino; and
R⁷ is hydrogen.

6. A compound according to claim 1 wherein together R⁵, R⁶ and R⁷ form two rings, having the structure: 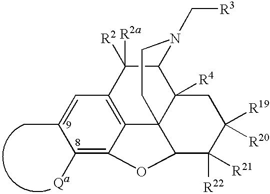
wherein
R³ is chosen from hydrogen, (C₁-C₇)hydrocarbon, heterocyclyl, and hydroxyalkyl;
R⁴ is hydrogen, hydroxy, amino or (C₁-C₆)alkoxy;
R¹⁹ is hydrogen or (C₁-C₆)alkyl;
R²⁰ is chosen from hydrogen, (C₁-C₆)alkyl and hydroxy((C₁-C₆)alkyl); or together, R¹⁹ and R²⁰ form a spiro-fused carbocycle of 5 to 10 carbons;
R²¹ is hydrogen;
R²² is chosen from hydroxy, (C₁-C₆)alkoxy and —NR¹³R¹⁴; or together, R²¹ and R²² form a carbonyl or a vinyl substituent; or together, R⁴ and R²¹ form a sixth ring.

7. A compound according to claim 6, wherein together, R⁴ and R²¹ form a sixth ring, of formula:

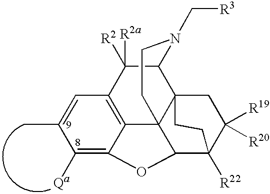

8. A morphinan according to claim 6, wherein R⁴ and R²¹ form a sixth ring, of formula 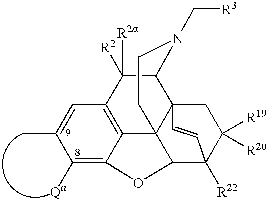
wherein
R¹⁹ is hydrogen;
R²⁰ is hydroxy((C₁-C₆)alkyl); and
R²² is (C₁-C₆)alkoxy.

9. A compound according to claim 1 having the formula 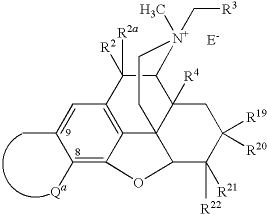
in which
R⁴ is hydrogen, hydroxy, amino or (C₁-C₆)alkoxy;
R¹⁹ is hydrogen or (C₁-C₆)alkyl;
R²⁰ is chosen from hydrogen, (C₁-C₆)alkyl and hydroxy((C₁-C₆)alkyl); or together, R¹⁹ and
R²⁰ form a spiro-fused carbocycle of 5 to 10 carbons;
R²¹ is hydrogen;
R²² is chosen from hydroxy, (C₁-C₆)alkoxy and —NR¹³R¹⁴; or together, R²¹ and R²² form a carbonyl or a vinyl substituent; and
E⁻ is a pharmaceutically acceptable anion.

10. A compound according to claim 1 wherein

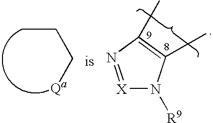

11. A compound according to any of claims 1-9 wherein 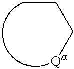
is chosen from

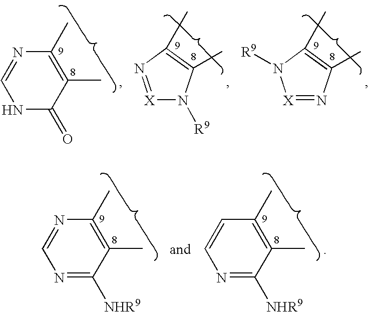

12. A compound according to any of claims 1-5 wherein 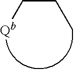
is chosen from

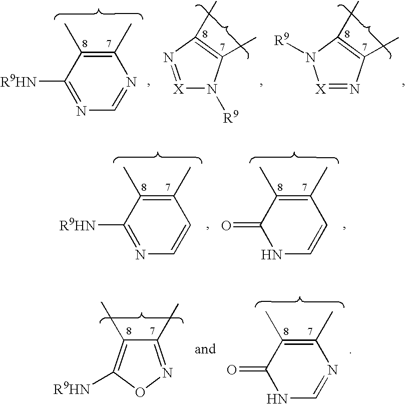

13. A compound according to claim 12 wherein R⁹ is chosen from hydrogen and (C₁-C₂₀)hydrocarbon.

14. A compound of formula 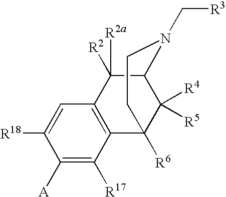
wherein
A is chosen from —C(═O)NR⁹R¹² and —C(═S)NR⁹R¹²;
R² and R^{2a} are both hydrogen or taken together R² and R^{2a} are ═O;
R³ is chosen from hydrogen, (C₁-C₈)hydrocarbon, heterocyclyl, heterocyclylalkyl and hydroxyalkyl;
R⁴ is chosen from hydrogen, hydroxy, amino, (C₁-C₆)alkoxy, (C₁-C₂₀)alkyl and (C₁-C₂₀)alkyl substituted with hydroxy or carbonyl;
R⁵ is (C₁-C₆)alkyl;
R⁶ is (C₁-C₆)alkyl;
or together R⁴, R⁵ and R⁶ may form from one to three rings, said rings having optional additional substitution;
R⁹ in each of its occurrences is independently chosen from H, alkyl and 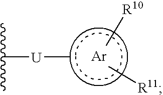
U is (CH₂)ₙ, wherein one or more CH₂ may be replaced by —O—, cycloalkyl or —CR^{1a}R^{1b};
R^{1a} and R^{1b} are chosen independently from hydrogen, halogen, (C₁-C₆)alkyl, (C₁-₆)alkoxy and (C₁-C₆)alkylthio;
Ar is an aryl or heteroaryl residue of one to three rings;
R¹⁰ is one or two residues chosen independently from hydrogen, hydroxyl, halogen, (C₁-C₆)alkyl, (C₁-C₆)alkoxy, halo(C₁-C₆)alkyl and halo(C₁-C₆)alkoxy and (C₁-C₆)alkylthio;
R¹¹ is H or 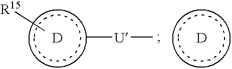
is an aryl or heteroaryl residue of one to three rings;
U′ is (CH₂)ₘ, wherein one or more CH₂ may be replaced by —O—, cycloalkyl, —CR^{1a}R^{1b}, —C(═O)— or —NH—;
R¹² is chosen from hydrogen and (C₁-C₆)alkyl;
R¹⁵ is one or two residues chosen independently from hydrogen, hydroxyl, halogen, (C₁-C₆)alkyl, (C₁-C₆)alkoxy, halo(C₁-C₆)alkyl and halo(C₁-C₆)alkoxy and (C₁-C₆)alkylthio;
one of R¹⁷ or R¹⁸ is NHR⁹ and the other is hydrogen;
m is zero or an integer from 1 to 6; and
n is an integer from 1 to 6.

15. A compound according to claim 14 wherein
A is —C(═O)NH₂;
R² and R^{2a} are hydrogen;
R³ is chosen from methyl, cyclopropylmethyl, cyclobutylmethyl, allyl and tetrahydrofuranylmethyl;
R⁴ is hydrogen;
R⁵ is methyl; and
R⁶ is methyl or ethyl.

16. A pharmaceutical formulation comprising a pharmaceutically acceptable carrier and a compound according to any of claim 1-9, 14 or 15.

17. A method for treating a disease or condition by altering a response mediated by an opioid receptor comprising bringing into contact with said opioid receptor a compound having the formula 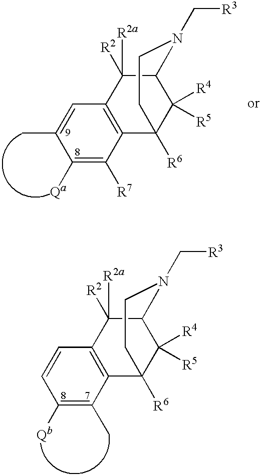
as defined in claim 1.

18. A method for treating a disease or condition by altering a response mediated by an opioid receptor comprising bringing into contact with said opioid receptor a compound having the formula 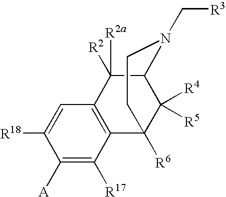
as defined in claim 14.

19. A method for treating a disease or condition by altering a response mediated by an opioid receptor comprising bringing into contact with said opioid receptor a compound having the formula 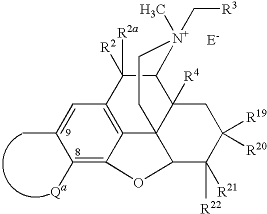
as defined in claim 9.

20. A method according to claim 17 or 18 wherein said disease or condition is chosen from the group consisting of pain, pruritis, diarrhea, irritable bowel syndrome, gastrointestinal motility disorder, obesity, respiratory depression, convulsions, coughing, hyperalgesia and drug addiction.

21. A method according to claim 19 wherein said condition is chosen from opioid-induced constipation and opioid-induced urinary retention.
